(19) United States
(12) Patent Application Publication
(10) Pub. No.: US 2025/0129434 A1
(43) Pub. Date: Apr. 24, 2025

(54) PLASMA CELL-FREE DNA SEQUENCING ASSAY FOR ADVANCED PROSTATE CANCER

(71) Applicant: Regents of the University of Minnesota, Minneapolis, MN (US)

(72) Inventors: Scott Dehm, Minneapolis, MN (US); Todd Knutson, Minneapolis, MN (US)

(21) Appl. No.: 18/901,167

(22) Filed: Sep. 30, 2024

Related U.S. Application Data
(60) Provisional application No. 63/541,175, filed on Sep. 28, 2023.

(51) Int. Cl. C12Q 1/6886

(52) U.S. Cl. CPC C12Q 1/6886; C12Q 2600/118; C12Q 2600/156

(57) ABSTRACT

Provided herein is a circulating tumor DNA assay for plasma cell-free DNA that can be used to prognosticate outcomes of metastatic castration-resistant prostate cancer (mCRPC) patients for being treated with androgen receptor (AR) inhibitors.

### CROSS REFERENCE TO RELATED APPLICATIONS

This application claims priority to U.S. Provisional Application No. 63/541,175, filed Sep. 28, 2023. The entire content of the application referenced above is hereby incorporated by reference herein.

### STATEMENT REGARDING FEDERALLY SPONSORED RESEARCH

This invention was made with government support under CA256157, and CA174777 awarded by National Institutes of Health. The government has certain rights in the invention.

### BACKGROUND

Standard of care treatments for patients with advanced prostate cancer include therapies that inhibit the androgen receptor (AR), a transcription factor essential for the homeostasis and survival of prostate cancer cells (1, 2). However, the disease will eventually progress to castration resistant prostate cancer (CRPC), which is a disease state responsible for nearly all prostate cancer deaths. Because the majority of CRPC remains AR dependent (3), first-line treatments for CRPC are potent AR-targeted therapies such as enzalutamide and abiraterone, which bind and inhibit the AR directly (e.g., enzalutamide) or prevent androgen synthesis (e.g., abiraterone) (4, 5). However, approximately 20-30% of CRPC patients have primary resistance to these agents and may instead benefit from AR-independent therapies. For instance, taxane chemotherapy or radium-223 have proven survival benefits in CRPC patients (6-9). Additionally, specific genomic features of CRPC tumors can be used to match patients to treatment with targeted therapies, such as poly (ADP-ribose) polymerase (PARP) inhibitors for CRPC with alterations in genes encoding homologous recombination DNA repair machinery (10-12), or the immune checkpoint inhibitor pembrolizumab for CRPC with high microsatellite instability or DNA mismatch repair deficiency (13). Optimizing treatment regimens with these and other AR-independent agents, as well as designing efficient clinical trials to test emerging new therapies, may be aided by prognostic markers that improve on currently available clinical prognostic models (14, 15) to identify CRPC patients unlikely to receive durable benefit from abiraterone and/or enzalutamide. Accordingly, there is currently a need for assays and related methods to provide improved diagnostic information for patients with prostate cancer. In particular, there is an unmet need for cfDNA assays able to prognosticate outcomes in metastatic castration-resistant prostate cancer (mCRPC patients), particularly in the context of AR-targeted therapies.

### SUMMARY

Accordingly, in one aspect the present invention provides a method for determining whether a prostate cancer patient should be treated with an androgen receptor (AR) independent therapy, comprising assaying a cell free DNA plasma sample from the patient to determine whether the sample comprises copy number gain or gain of function mutations to MYC, MYCN and/or AR, wherein the presence of said copy number gain or gain of function mutations to MYC, MYCN and/or AR indicates that the patient should be treated with an AR-independent therapy.

### BRIEF DESCRIPTION OF DRAWINGS

FIG. 1. Cell free DNA (cfDNA) yield and circulating tumor DNA (ctDNA) fraction in A031201 plasma specimens. (1a) CONSORT flow diagram of patients enrolled in A031201 and plasma cfDNA specimens analyzed by targeted DNA-seq. (1b) AR-ctDETECT targeted DNA-seq assay design. (1c) Scatterplot of ctDNA aneuploidy fraction estimated by ichorCNA using off-target DNA-seq reads vs. cfDNA yield from n=776 plasma specimens analyzed by targeted DNA-seq. Loess trendline and 95% confidence intervals are shown. Violin plots illustrate data density from min to max. Boxes in violin plots illustrate median and interquartile range. Whiskers illustrate 1.5× interquartile range. (1d) ctDNA aneuploidy fraction estimate as in (1c) using cfDNA isolated from pooled plasma from healthy donors. An assay cutoff of mean+3 standard deviations (0.142) distinguishes ctDNA aneuploidy-high specimens.

FIG. 2. Comprehensive profiling of AR genomic alterations in A031201 cfDNA. (2a) ctDNA aneuploidy fraction estimated by ichorCNA in targeted DNA-seq data from n=776 cfDNA specimens. Samples below the ctDNA aneuploidy-high threshold of 0.142 are in gray. (2b) Relative AR gene copy number ratio in cfDNA specimens ordered left to right as in (2a). A log 2 ratio>=0.3 is considered a gain. (2c) Relative AR upstream enhancer copy number ratio in cfDNA specimens ordered left to right as in (2a). A log 2 ratio>=0.3 is considered a gain. (2d) Number of AR gene structural rearrangements (AR-GSRs) in cfDNA specimens ordered left to right as in (2a). AR-GSRs predicted to encode AR variant proteins with a truncated ligand binding domain (LBD), and AR-GSRs predicted to be associated with AR amplification on extrachromosomal DNA (ecDNA), are shown. (2e) AR single nucleotide variants (SNVs) detected in cfDNA specimens as in (2a). (2f) Scatterplot of relative AR gene copy number ratio and number of AR-GSRs. Dark denotes samples with 2 or more AR-GSRs and AR gene body copy gain, which is a signature of AR ecDNA (shown also in (2d)). (2g, 2h) Forest plots illustrating hazard ratio and 95% confidence intervals for (2g) radiographic progression (rPFS) and (2h) overall survival (OS) in patients demonstrating indicated cfDNA features. Multivariable analysis is adjusted for ctDNA aneuploidy fraction. P-values are adjusted for multiplicity using the Benjamini-Hochberg method (false discovery rate). A FDR <0.05 is considered statistically significant. (2i) UpSet plot showing co-occurrence of indicated AR genomic alterations. (2j) Scatterplot of relative AR gene copy number ratio and AR upstream enhancer copy number ratio. Dots are based on copy number gain of the AR gene body only, AR upstream enhancer only, or both the AR gene body and AR upstream enhancer.

FIG. 3. MYC and MYCN copy number gains in A031201 cfDNA. (3a) Relative MYC and MYCN gene copy number ratios in cfDNA specimens based on ctDNA aneuploidy fraction estimated by ichorCNA (samples below the ctDNA aneupoidy-high threshold of 0.142 are in gray/pink). A log 2 ratio>=0.3 is considered a gain. (3b) UpSet plot showing co-occurrence of MYC and MYCN copy number gains in cfDNA samples from (3a). (3c) UpSet plot showing co-occurrence of MYC and MYCN copy number gains in mCRPC biopsy specimens from 444 patients analyzed in the AACR-PCF Stand Up To Cancer East Coast study. (3d) UpSet plot showing co-occurrence of MYC and MYCN copy number gains in mCRPC biopsy specimens from 101 patients analyzed in the AACR-PCF Stand Up To Cancer West Coast study.

FIG. 4. Prevalent genomic alterations in ctDNA-positive A031201 cfDNA. (4a) cfDNA specimens were stratified into 3 groups based on samples exceeding a ctDNA aneuploidy threshold (ctDNA aneuploidy-high Group 1), samples below the ctDNA aneuploidy threshold but harboring a likely somatic pathogenic mutation, an AR-GSR, and/or copy number gain of AR, MYC or MYCN (ctDNA aneuploidy-low Group 2), or samples below the ctDNA aneuploidy threshold and lacking a likely somatic pathogenic mutation, an AR-GSR, or copy number gain of AR, MYC or MYCN (ctDNA-negative Group 3). (4b) Pie charts illustrating frequency of AR or MYC/MYCN alterations detected in ctDNA aneuploidy-high Group 1 or ctDNA aneuploidy-low Group 2. (4c) TP53, PTEN, and RB1 single nucleotide variants (SNVs) detected in ctDNA-positive cfDNA specimens ordered left to right as in (a). (4d, 4e, 4f) Relative (4d) TP53, (4e) PTEN, or (4f) RB1 gene copy number ratios in cfDNA specimens ordered left to right as in (4a). A log 2 ratio <=−0.3 in a ctDNA-positive sample is considered a loss. (4g, 4h, 4i) UpSet plots showing relationships between (4g) TP53, (4h) PTEN, or (4i) RB1 SNVs and copy number losses in ctDNA-positive cfDNA samples. (4j) Pie charts illustrating frequency of TP53, PTEN, or RB1 alterations detected in ctDNA aneuploidy-high Group 1 or ctDNA aneuploidy-low Group 2. (4k) UpSet plot showing relationships between alterations in TP53, PTEN, and RB1 in ctDNA-positive cfDNA specimens.

FIG. 5. Pathogenic mutations detected in A031201 ctDNA. Oncoprint of single nucleotide variants (SNVs) occurring in indicated genes across ctDNA specimens in ctDNA aneuploidy-high Group 1 and ctDNA aneuploidy-low Group 2. The frequency of SNVs within these groups is indicated at the right. Samples are ordered left to right by descending ctDNA aneuploidy fraction estimate.

FIG. 6. Survival of ctDNA-positive A031201 patients. (4a, 4b) Kaplan-Meier plots of (4a) radiographic progression-free survival and (4b) overall survival in patients classified as ctDNA-positive or ctDNA-negative. (4c, 4d) Kaplan-Meier plots of (4c) radiographic progression-free survival and (4d) overall survival in patients classified as belonging to ctDNA aneuploidy-high Group 1, ctDNA aneuploidy-low Group 2, or ctDNA-negative Group 3.

### DETAILED DESCRIPTION

Liquid biopsy assays tailored for mCRPC patients are needed to detect ctDNA, identify molecular alterations, and predict survival in the context of androgen receptor (AR) inhibitors. This study develops a novel cell-free plasma DNA sequencing assay customized for mCRPC patients that detects critical molecular alterations that occur even in otherwise ctDNA-negative patients. Whether this assay has prognostic utility as a liquid biopsy tool using plasma specimens collected from patients enrolled in the randomized phase III clinical trial that evaluated enzalutamide vs. enzalutamide plus abiraterone was investigated. It was determined that detecting ctDNA using this assay is associated with adverse prognosis and enables identification of key molecular alterations in targeted genes. These findings offer clinical utility for personalized treatment decisions in mCRPC patients.

Liquid biopsy assays are useful to prognosticate outcomes of metastatic castration-resistant prostate cancer (mCRPC) patients treated with androgen receptor (AR) inhibitors. As described herein, an assay has been developed to interrogate AR and a panel of mCRPC driver genes to detect circulating tumor DNA (ctDNA) in limiting plasma cell-free DNA (cfDNA) available from mCRPC patients in the Alliance A031201 randomized phase 3 trial of enzalutamide with or without abiraterone acetate and prednisone. Of 776 patients with evaluable cfDNA sequencing data, 59% were ctDNA-positive, with 26% having high ctDNA aneuploidy and 33% having low ctDNA aneuploidy but displaying AR gain or structural rearrangement, MYC/MYCN gain, or a pathogenic mutation. ctDNA-positive patients had significantly worse median overall survival than ctDNA-negative patients (29.0 months vs. 47.4 months, respectively). Herein, it is shown that mCRPC patients identified as ctDNA-positive using the assay had poor survival despite treatment with potent AR inhibition in a phase 3 trial.

Accordingly, as described herein, a circulating tumor DNA (ctDNA) assay for plasma cell-free DNA (cfDNA) that can prognosticate outcomes of metastatic castration-resistant prostate cancer (mCRPC) patients being treated with androgen receptor (AR) inhibitors has been developed. In certain embodiments, the invention is directed to such assays and to the uses of such assays in treatment and diagnosis.

A targeted cfDNA-sequencing assay was designed to detect mCRPC-specific alterations and used to investigate evaluable plasma specimens from mCRPC patients (n=790) enrolled in a randomized phase III trial of enzalutamide with or without abiraterone acetate and prednisone. ctDNA positivity was defined as >20% ctDNA using a traditional aneuploidy-based estimate (Group 1) or a novel definition of ≤20% ctDNA, but with detection of alterations in AR, MYC and/or MYCN (Group 2). Kaplan-Meier estimates and log-rank tests were employed for survival analysis.

Of 776 patients with evaluable cfDNA sequencing data, 480 (62%) were ctDNA-positive, with 324 (42%) in Group 1 and an additional 156 (20%) in Group 2 with AR, MYC, and/or MYCN alterations detected. ctDNA-positive patients in Groups 1 and 2 had similarly poor median overall survival (OS) of 25.4 months (95% CI: 21.8-29.0) and 29.7 months (95% CI: 27.3-35.5), respectively, as compared to ctDNA-negative patients who had a median OS of 49.1 months (95% CI: 42.3-53.5). Relative to ctDNA-negative patients, OS was inferior in ctDNA-positive patients in Groups 1 (HR 2.3, 95% CI: 1.9-2.9, P<0.0001) and Group 2 (HR 1.8, 95% CI: 1.5-2.3, P<0.0001).

Patients identified as ctDNA-positive using a mCRPC-specific ctDNA sequencing assay had poor survival despite treatment with potent AR inhibition in a phase III trial. Other assays for cell free DNA-sequencing with plasma cfDNA isolated from CRPC patients require much higher levels of input cfDNA and have not been shown to stratify patients treated in a phase III trial by overall survival.

Accordingly, provided herein is a method for determining whether a prostate cancer patient should be treated with an androgen receptor (AR) independent therapy, comprising assaying a cell free DNA plasma sample from the patient to determine whether the sample comprises copy number gain or gain of function mutations to MYC, MYCN and/or AR, wherein the presence of said copy number gain or gain of function mutations to MYC, MYCN and/or AR indicates that the patient should be treated with an AR-independent therapy.

In certain embodiments, the method is performed prior to the patient being treated with an AR inhibitor.

In certain embodiments, the method is performed while the patient is being treated with an AR inhibitor.

In certain embodiments, the method is performed after the patient has been treated with an AR inhibitor.

In certain embodiments, the AR inhibitor is abiraterone, enzalutamide, apalutamide or darolutamide.

In certain embodiments, the AR inhibitor is abiraterone.

In certain embodiments, the AR inhibitor is enzalutamide.

In certain embodiments, the AR inhibitor is apalutamide.

In certain embodiments, the AR inhibitor is darolutamide.

In certain embodiments, the assaying determines a copy number gain of the AR gene.

In certain embodiments, the assaying determines an AR upstream enhancer gain.

In certain embodiments, the assaying determines a copy number gain of the AR gene body and an AR upstream enhancer gain.

In certain embodiments, the assaying determines an AR mutation that encodes a H875Y substitution in the AR protein.

In certain embodiments, the assaying determines an AR gene structural rearrangement (AR-GSR).

In certain embodiments, the assaying determines the presence of copy number gain or gain of function mutations to MYC, MYCN and AR.

In certain embodiments, the assaying further determines a mutation to TP53, PTEN, and/or RB1.

In certain embodiments, the patient has an estimated ctDNA aneuploidy fraction of <=about 14.2%.

Also provided are systems and assays for performing the methods described herein, including a system or assay that determines or is used to determine whether a prostate cancer patient should be treated with an androgen receptor (AR) independent therapy, the system or assay comprising means to assay a cell free DNA plasma sample from the patient to determine whether the sample comprises copy number gain or gain of function mutations to MYC, MYCN and/or AR. The presence of said copy number gain or gain of function mutations to MYC, MYCN and/or AR indicates that the patient should be treated with an AR-independent therapy.

In certain embodiments, the assay determines or is used to determine a copy number gain of the AR gene.

In certain embodiments, the assay determines or is used to determine an AR upstream enhancer gain.

In certain embodiments, the assay determines or is used to determine a copy number gain of the AR gene body and an AR upstream enhancer gain.

In certain embodiments, the assay determines or is used to determine an AR mutation that encodes a H875Y substitution in the AR protein.

In certain embodiments, the assay determines or is used to determine an AR gene structural rearrangement (AR-GSR).

In certain embodiments, the assay determines or is used to determine the presence of copy number gain or gain of function mutations to MYC, MYCN and AR.

In certain embodiments, the assay further determines or is used to determine a mutation to TP53, PTEN, and/or RB1.

Prostate cancer is the second leading cause of cancer deaths among US men. Prostate cancer is heterogeneous in all stages of disease both in terms of clinical behavior and morphology. For patients with localized prostate cancer, this heterogeneity can often be overcome with curative treatments including radical prostatectomy or radiation therapy. However, for patients with metastatic prostate cancer, this disease heterogeneity represents a formidable clinical challenge. Initially, metastatic prostate cancer can be controlled by endocrine therapies that inhibit the androgen receptor (AR), a master regulator transcription factor in cells of prostatic lineage. Despite the benefit from these endocrine therapies, approximately 20% of men have primary resistance, while acquired resistance occurs in most men within 1-2 years. This stage of the disease, referred to as metastatic castration resistant prostate cancer (mCRPC) is responsible for nearly all prostate cancer deaths. There is an urgent need for robust prognostic and predictive diagnostics to address the challenge of disease heterogeneity for men with mCRPC. The work described herein demonstrates that key genomic alterations in AR and non-AR pathways can be measured non-invasively in patients and incorporated into clinical-genomic prognostic and predictive models of clinical outcomes of first-line AR inhibitor therapy in men with mCRPC.

The androgen receptor (AR), also known as NR3C4 (nuclear receptor subfamily 3, group C, member 4), is a type of nuclear receptor that is activated by binding any of the androgenic hormones, including testosterone and dihydrotestosterone, in the cytoplasm and then translocating into the nucleus. In humans, the androgen receptor is encoded by the AR gene located on the X chromosome at Xq11-12.

The androgen receptor (AR) is a steroid receptor transcriptional factor for testosterone and dihydrotestosterone that has four main domains, the N-terminal domain, DNA-binding domain, hinge region, and ligand-binding domain. AR plays pivotal roles in prostate cancer, especially castration-resistant prostate cancer (CRPC). Androgen deprivation therapy can suppress hormone-naïve prostate cancer, but prostate cancer changes AR and adapts to survive under castration levels of androgen. These mechanisms include AR point mutations, AR overexpression, changes of androgen biosynthesis, constitutively active AR splice variants without ligand binding, and changes of androgen cofactors.

The main function of the androgen receptor is as a DNA-binding transcription factor that regulates gene expression; [13] however, the androgen receptor has other functions as well. [14] Androgen-regulated genes are critical for the development and maintenance of the male sexual phenotype.

A key concept in the field is that the majority of tumors progressing to castration resistance remain dependent on AR signaling. Therefore, front-line therapies for mCRPC are newer, more potent endocrine therapies such as enzalutamide and abiraterone, which bind and inhibit the AR ligand binding domain (enzalutamide) or prevent synthesis of the AR ligands testosterone and dihydrotestosterone (abiraterone). However, clinical cross-resistance between these and other current AR inhibitors is a major problem, with only a minority of patients responding to enzalutamide after abiraterone therapy or vice versa, and usually for only short periods of time. Additionally, approximately 20-30% of mCRPC patients display de novo resistance to either of these agents as front-line therapies, and these patients would benefit from alternative approaches. Taxane chemotherapy and radium-223 also have proven survival benefits in mCRPC patients, and target pathways independent of AR. Optimizing sequencing regimens with these and other AR-independent agents, as well designing efficient clinical trials to test emerging new therapies including immunotherapies, chemotherapy combinations, PARP inhibitors, or novel AR pathway inhibitors, will require prognostic and predictive diagnostics that can identify patients that are unlikely to receive durable benefit from abiraterone and/or enzalutamide.

The complexity of resistance exists in the context of disease heterogeneity within and between patients with wide variation in genetic and genomic profiles, prior therapy, symptoms, and patterns of metastatic spread. Lineage plasticity has emerged as one mechanism of resistance in which dependence on AR activity is lost or reduced and a transition to a small cell/neuroendocrine phenotype becomes dominant. These patients often display coordinate loss of prostate cancer tumor suppressor genes RB1 and TP53, as well as copy gain of the MYCN oncogene. However, the majority (>80%) of cases display resistance accompanied by rises in serum levels of the PSA protein (which is under transcriptional control by AR), thus providing strong clinical support that AR activity persists in these tumors. Reactivation of AR transcription in mCRPC can be driven by: 1) AR overexpression caused by copy gain of the AR gene body and/or an genomic enhancer upstream of AR, 2) expression of truncated AR splice variants such as AR-V7 that function as constitutively-active transcription factors, 3) genomic structural rearrangements in the AR gene (AR-GSRs), which can drive expression of AR variants that are molecularly distinct from AR-V7, 4) production of intratumoral androgens, 5) acquisition of somatic AR mutations in the ligand binding domain that broaden the repertoire of ligands that can activate AR, and 6) up-regulation of the glucocorticoid receptor (which shares many transcriptional targets with AR).

It is herewith proposed to capture this biologic diversity through the novel development of a clinical-genomic model that combines patient phenotypic factors such as tumor burden and pattern of spread with genetic drivers of AR therapy resistance to optimally identify and predict therapeutic outcomes in men with mCRPC prior to treatment initiation with an AR inhibitor. This predictive model would be significant in its ability to identify such men who have suboptimal responses to AR inhibition and who are thus suitable for new treatment approaches.

Circulating tumor DNA (ctDNA) is found in the bloodstream and refers to DNA that comes from cancerous cells and tumors. Most DNA is located inside a cell's nucleus. However, as cancerous cells die, the dead cells are broken down and their contents, including DNA, are released into the bloodstream. ctDNA are these small pieces of DNA. The quantity of ctDNA varies among individuals and depends on the type of tumor, its location, and for cancerous tumors, the cancer stage.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” as used herein includes mammals, fish, birds, reptiles, and amphibians.

The term “mammal” as used herein refers to humans, higher non-human primates, rodents, domestic, cows, horses, pigs, sheep, dogs and cats. In one embodiment, the mammal is a human, e.g., a human male or human female. The term “patient” as used herein refers to any animal including mammals. In one embodiment, the patient is a mammalian patient. In one embodiment, the patient is a human patient, e.g., a human male or human female patient.

The invention will now be illustrated by the following non-limiting Example.

### EXAMPLES

### Example 1. Prognostic Utility of an AR-Informed Circulating Tumor DNA Assay for Men with Metastatic Castration-Resistant Prostate Cancer in a Phase III Trial

Currently, there is an unmet need for cfDNA assays able to prognosticate outcomes in mCRPC patients, particularly in the context of AR-targeted therapies.

Based on this need, a targeted cfDNA sequencing (cfDNA-seq) assay was developed to interrogate alterations in AR and other known actionable alterations that occur in mCRPC patients, using low input volumes of plasma. The determination of ctDNA positivity can be accomplished through detection of aneuploidy and/or mutations present in cancer but absent in normal cells. A unique aspect of the assay design is the ability to identify ctDNA uniquely in mCRPC patients via comprehensive profiling of AR alterations including mutations, amplification, enhancer amplification, and gene structural rearrangements (GSRs), as well as gains in MYC and/or MYCN, due to the high sensitivity for detecting these alterations relative to normal cells. To evaluate the clinical utility of this cfDNA-seq assay for prognosticating outcomes in mCRPC patients, plasma collected prior to treatment in the phase 3 Alliance A031201 trial, which randomized men 1:1 to treatment with enzalutamide or enzalutamide plus abiraterone, was studied. While this trial did not demonstrate improved survival with the combination therapy, it represented an important opportunity to evaluate the assay described herein in both treatment arms for prognostic utility.

In this work, mCRPC-specific genomic alterations are detected at high frequency in cfDNA specimens classified as having high ctDNA aneuploidy. These patients were assigned to a ctDNA aneuploidy-high Group 1 and demonstrated they have a short duration of radiographic progression-free survival (rPFS) and overall survival (OS) compared with patients assigned to a ctDNA-negative Group 3. In patients with low ctDNA aneuploidy, a second ctDNA-positive group was identified on the basis of detectable pathogenic tumor-derived mutations, AR-GSRs, and/or copy gains in AR, MYC or MYCN. These patients were assigned to a ctDNA aneuploidy-low Group 2, and demonstrated they also have shorter rPFS and OS compared with patients in ctDNA-negative Group 3. These data validated the importance of evaluating these genomic alterations in patients having low ctDNA aneuploidy in a phase 3 trial context, as well as the prognostic utility of detecting ctDNA in mCRPC patients being treated with contemporary AR-targeted therapies.

### Patients and Methods

### Patients and Plasma Samples

Alliance A031201 (Clinicaltrials.gov: NCT01949337) was a randomized phase 3 clinical trial conducted by the National Cancer Institute (NCI)-funded NCTN Alliance for Clinical Trials in Oncology to compare the anticancer effects of first-line enzalutamide with or without abiraterone acetate and prednisone in men with mCRPC. Men with previously untreated mCRPC and progressive metastatic disease despite ongoing androgen deprivation therapy (ADT) were included. Prior docetaxel or first generation AR inhibitors were permitted in earlier settings. Patients were treated with standard of care doses of enzalutamide 160 mg/d with or without abiraterone acetate and prednisone (1:1 randomization, open label) until clinical or radiographic progression and patients were followed long-term for mortality. Details of the patients, inclusion criteria, study endpoints, and specimens collected for the correlative analysis have been described previously (31).

Cell-Free DNA (cfDNA) Isolation and Yield

Cell-free DNA (cfDNA) was isolated from 1-3 mL (mean=2.12 mL, standard deviation=0.49 mL) of input plasma using a Circulating Nucleic Acid kit (Qiagen catalog 55114) according to the manufacturer's recommendation. cfDNA samples were eluted in 30-50 μL of elution buffer. Concentration was determined from 3 μL input cfDNA using a Qubit 1X dsDNA HS Assay kit (Thermo Fisher catalog Q33231) and a Qubit Fluorometer (Thermos Fisher catalog Q33216). cfDNA yield was calculated by dividing the total mass of cfDNA eluted by the volume of plasma used as input.

Targeted DNA-Sequencing of cfDNA

DNA-seq library preparation and sequencing was performed by the University of Minnesota Genomics Center (UMGC). Concentrations of cfDNA samples were determined using Quant-iT PicoGreen dsDNA Assay (ThermoFisher catalog P7589) per manufacturer's recommendations. An initial 38 cfDNA samples with masses of at least 1 ng were prepared as DNA-seq libraries using a ThruPLEX Tag-Seq kit (Takara, Catalog R400586) according to manufacturer's recommendations. A subsequent 95 cfDNA samples with masses of at least 5 ng were prepared as DNA-seq libraries using a ThruPLEX Tag-Seq HV kit (Takara Catalog R400743) according to manufacturer's recommendations. All remaining cfDNA samples with masses of at least 1 ng were prepared as DNA-seq libraries using a ThruPLEX Plasma-Seq kit (Takara, Catalog R400681) with Takara Bio DNA unique dual index kits tubes A (Takara, Catalog R400665), B (Takara, Catalog R400666), C (Takara, Catalog R400667), and D (Takara, Catalog R400668) according to manufacturer's recommendations. Libraries were cleaned using 1X Ampure XP beads (Beckman Coulter, Catalog A63880) followed by DNA quantification. Targeted DNA-seq was performed using SureSelect (Agilent Technologies) capture protocol (Version B.3 Jun. 2015) with minor modifications. Library input was between 750 ng-1 μg. For libraries with masses below this range, a second amplification step was performed using KAPA HiFi HotStart Ready Mix (Roche), following manufactured protocols and a second 1X Ampure XP bead clean-up to increase concentrations. Libraries were hybridized overnight (between 16-24 hours) using a SureSelect XT Reagent Kit (Agilent, Catalog 930672) and SureSelect XT Custom Baits (Agilent, Catalog 5191-6908). Post hybridization libraries were amplified by PCR using KAPA HiFi HotStart Ready mix and library amplified primer mix (Roche, Catalog KK2621). Post capture, libraries were cleaned using 1× Ampure XP beads. Libraries were quantified and pooled in equimolar amounts for sequencing using an Illumina NovaSeq 6000 SP flow cell with 2×150 bp settings. Data are available via dbGaP (accession number phs003325.v1.p1)

### DNA-Seq Read Mapping and Duplicate Removal

Adapters and poor-quality bases were trimmed from paired-end FASTQ files using Trimmomatic (v. 0.39). An initial pilot experiment with 1.3 to 30 ng of cfDNA extracted from 38 plasma specimens was used for library generation using unique molecular index (UMI) sequences. These 38 samples were processed with umi-tools (v. 1.1.2) to identify, correct, and remove UMIs from the reads and place them into the BAM RX tag. Reads were mapped against the human reference genome (GRCh37/hg19) using bwa mem (v. 0.7.17) (http://arxiv.org/abs/1303.3997) and samblaster (v. 0.1.24) was used to limit the number of split alignments for a read (max=2) and limit the number of non-overlapping base pairs between two alignments (min=20). Read duplicate marking and removal was explored using four strategies: Picard tools (v. 2.25.5) https://broadinstitute.github.io/picard/) MarkDuplicates (defaults), Picard MarkDuplicates with the BARCODE_TAG option set to RX, Picard UmiAwareMarkDuplicatesWithMateCigar with the BARCODE_TAG option set to RX, or collapsing duplicates to a single consensus read via FgBio (v. 1.3.0) functions SetMateInformation, GroupReadsByUmi (using RX tag), CallMolecularConsensusReads, FilterConsensusReads, and remapping deduplicated consensus reads with bwa mem. Use of UMI barcodes for removal of PCR duplicates only improved the number of unique total, mapped, and properly paired DNA-seq reads by an average of 3-4% compared to removal of PCR duplicates using a UMI-independent method. This average improvement in the number of unique DNA-seq reads was mainly attributed to 5 of the 38 pilot cfDNA samples with the highest input mass, indicating that use of UMIs did not enhance the analysis of cfDNA samples with low input masses to a meaningful extent. Based on these results, UMI-independent library preparation and PCR duplicate removal methods were used for analysis of subsequent samples. For these libraries that did not contain UMIs, Picard MarkDuplicates was used for read duplicate removal. Read sequencing, mapping, and capture metrics were collected from samtools (v 1.12) flagstat and Picard tools functions (CollectInsertSizeMetrics, CollectAlignmentSummaryMetrics, CollectHSMetrics, CollectGCBiasMetrics).

Estimation of ctDNA Aneuploidy Fraction

The circulating tumor DNA (ctDNA) aneuploidy fraction was estimated by the ichorCNA R package (v.0.2.0) with R (v.4.2.0) using off-target reads as input for ichorCNA as described previously (https://github.com/GavinHaLab/ichorCNA_offtarget). Specifically, duplicates-removed bam files were filtered with bedtools (v. 2.3.0) intersect software to remove all reads overlapping the targeted regions, leaving only off-target reads (i.e. approximating low-pass whole genome DNA-seq) for coverage analysis. The number of mapped reads within non-overlapping 1 Mb windows was determined using the readCounter function from HMM Copy Utils software library (commit: 5911bf6, https://github.com/shahcompbio/hmmcopy_utils). Only reads with mapping quality scores >20 were included. These genome-wide binned read count values were used as input to the runIchorCNA.R script. It was executed using custom initialization parameters for ploidy (2, 3) and normal fraction levels (0.5, 0.6, 0.7, 0.8, 0.9). Otherwise, the script used default settings (including the use of package extra data files: gc_hg19_1000 kb.wig, map_hg19_1000 kb.wig, GRCh37.p13_centromere_UCSC-gapTable.txt, and HD_ULP_PON_1 Mb_median_normAutosome_mapScoreFiltered_median.rds).

### SNV/Indel Calling and Filtering

For each sample, Freebayes (v.1.3.1) (http://arxiv.org/abs/1207.3907) and GATK MuTect2 (v.4.1.6.0) were used to identify SNV/indel variants using the duplicates-removed BAM file as input. Freebayes was used with default parameters, except the minimum allele frequency was set to 0.01, minimum mapping quality set to 20, and minimum number of alternate allele reads set to 3. MuTect2 was used with default parameters, except a gnomAD germline resource file of known SNPs was used to filter common variants (gnomad.exomes.r2.0.2.sites), the MateOnSameContigOrNoMappedMateReadFilter filtering was disabled, and the minimum allele frequency was set to 0.0000025 for alleles not found in the germline reference file. Variant calling in Freebayes and MuTect2 was restricted to GRCh37/hg19 regions targeted by the capture probes. Output files were processed by the rtg-tools (v.3.11) vcfdecompose function designed to break large indels and multiple-nucleotide polymorphisms into smaller sized SNPs. Variants were normalized with vt software (v.0.57) and rtg-tools vcffilter was used to retain only variants with 6 or more reads containing the alternative allele.

The rtg-tools vcfeval function, with decompose and squash-ploidy parameters enabled, was used to compare filtered Freebayes and MuTect2 VCFs and intersecting calls were retained. Variants were annotated with vcf2maf (v 1.6.21) (DOI: 10.5281/zenodo.1185418), which depends on the Ensembl Variant Effect Predictor (v. 104.3), and OncoKB (v. 3.3.1). Variants from all intersecting and non-intersecting comparisons were imported into R where each was labeled, filtered, and counted using custom scripts. SNV/indels were considered true positives if they were identified by both Freebayes and MuTect2 and met the following criteria: (1) variant was curated in OncoKB and must be labeled Oncogenic or Likely Oncogenic; or (2) if variant was not curated in OncoKB, it must labeled Oncogenic or Likely Oncogenic, but must also not have a splice-site or splice-region classification in OncoKB. Additional filtering criteria was applied for certain genes based on known germline or non-pathogenic variant status: PMS2 (remove p.Arg20Gln); MET (remove p.Glu168Asp); KMTC2 (remove p.Tyr816Ter); ATM (remove p.His1380Tyr); FANCA (remove p.Ser1088Phe and p.Ser858Arg); NCOR1 (remove p.Arg190Ter); AURKA (remove p.Phe31Ile); FOXA1 (ignore OncoKB filtering above and remove all synonymous mutations, p.Ala83Thr, p.Ser448Asn, and p.Leu148Val). Final filtered variants were classified as likely germline if they affected HSD3B1 or any gene other than AR, TP53, PTEN, ERF, PIK3CA, or CDK12 with a variant allele frequency between 0.45-0.55 or >0.95. Otherwise, filtered variants were classified as likely somatic.

### Identification of Gene-Specific Copy Number Alterations

Read depths were calculated by mosdepth (v. 0.3.3) using the duplicates-removed BAM file at every position targeted by the capture panel (i.e. gene exons or control regions) with no limit on the number of reads counted. Default parameters were used, except—fast-mode and—flag 1796, which discarded UNMAPPED, SECONDARY, QCFAIL, or DUPLICATE reads from the read depth sum at every position.

Copy number ratios were determined by comparing the normalized read depth from each sample against the average normalized read depth from 65 samples with tumor fraction≤0.05, as determined by ichorCNA. For each gene, the mean read depth was calculated across the concatenated target regions (e.g. all exons). The within-sample normalized read depth was calculated differently for genes found on chrX versus autosomes because one of the five control (C) regions is located on chrX: C1 (chr9: 98258995-98264381), C2 (chrX: 16153017-16159789), C3 (chr14: 105249980-105255049), C4 (chr15: 40514992-40520036) and C5 (chr15: 67390102-67395049). For genes on chrX, norm_read_depth=mean read coverage of chrX gene/median of [mean read coverage (C1), mean read coverage (C2)*2, mean read coverage (C3), mean read coverage (C4), mean read coverage (C5)]. For genes on autosomes: norm_read_depth=mean read coverage of autosome gene/median of [mean read coverage (C1)/2, mean read coverage (C2), mean read coverage (C3)/2, mean read coverage (C4)/2, mean read coverage (C5)/2]). Copy number ratios were calculated as: norm_read_depth for Gene X in sample Y/mean [norm_read_depth for Gene X in samples classified as low tumor fraction]. Genes were classified as copy number gain if the log 2 (copy number ratio) was >0.3 and copy number loss if the log 2 (copy number ratio) was <−0.3 and the sample was classified as ctDNA-positive.

### Identification of AR Gene Structural Rearrangements (AR-GSRs)

AR gene structural rearrangements (AR-GSRs) were called using Manta (v.1.5.0), DELLY (v.0.8.1), SvABA (v.1.1.0), and LUMPY (v.0.3.0). The complete BAM (PCR duplicates removed) was supplied as input to Manta, DELLY, and SvABA; whereas, a discordant reads only BAM and split reads only BAM was supplied as input to LUMPY. Manta was used in tumor-only, exome mode with default parameters, except minEdgeObservations and minCandidateSpanningCount was changed from 3 to 2 reads. The DELLY call function was used with default parameters, except the insert size cutoff, s, was changed from 9 to 15 (median+s*MAD, deletions only). Variants in telomeres, centromeres, and other low complexity regions were excluded via a bed file provided by the software developer. Variants labeled “IMPRECISE” in the INFO column were removed and only variants labeled “PASS” in the FILTER column were retained. SvABA was used in tumor-only mode with default parameters and the unfiltered.sv.vcf was used for downstream analysis. LUMPY-based calling required a histogram of observed library sizes to be generated by the pairend_distro.py script using the complete BAM as input. The breakpoint probability distortion was returned along with the mean and standard deviation of the library, which were supplied to the LUMPY function as parameters.

VCFs from each caller were converted from symbolic to breakend format (see VCF specification 4.2, section 5.4, https://github.com/samtools/hts-specs) using the rtg-tools (v.3.11) svdecompose function. Representing all SVs in breakend format allowed for across-caller VCF comparison. SvABA VCFs only represent SVs in breakend format and do not predict SVTYPE for any calls. For SvABA VCFs, SVTYPE (INV, DEL, DUP, or BND) was predicted using a custom R script that examined SV breakend orientation and mate pair information. For the other callers, the original SVTYPE was preserved in the decomposed breakend format using a custom R script. Any SVs with breakends on different chromosomes were re-labeled as translocations (TRA). The rtg-tools bndeval function, with tolerance parameter set to 1000 bp, was used to compare any two VCFs and intersecting calls were retained. Variants from all intersecting and non-intersecting comparisons were imported into R (v. 4.2.0) where each was labeled, filtered, and counted using custom scripts. SV length and read support values were parsed from data contained within each VCF record. SV length was calculated as the base-pair difference between breakends (TRA variants given length −1). The number of split or discordant reads supporting an SV call was extracted from the following VCF fields, respectively: Manta (FORMAT: SR, PR), DELLY (FORMAT: RV, DV), SvABA (FORMAT: SR, DR), and LUMPY (INFO: SR, PE). SVs were removed from further analysis if either breakend overlapped with a list of known artifactual mapping regions (bed file) discovered by the ENCODE project. AR-GSRs were considered true positives if at least one breakend fell within the AR gene region, was identified by at least two of four SV callers, and had at least 3 supporting split reads and 3 supporting discordant paired-end reads.

Each AR-GSR was evaluated for whether it was likely to encode an AR variant with a truncated ligand binding domain (LBD). Criteria for inclusion in this list of likely LBD-truncating AR-GSRs were 1) a DEL, INV, or BND that fully or partially overlaps AR exon 4, 5, 6, 7, and/or the start of exon 8 through the stop codon of exon 8, but does not overlap the interval spanning the beginning of AR exon 1 through the end of AR exon 3; or 2) a DUP, INV, or BND that fully overlaps the interval from the start of AR exon 1 through the end of AR exon 3 with a breakpoint anywhere between the end of AR exon 3 and the stop codon in AR exon 8; or 3) a DUP with both start and end breakpoints within the AR gene and flanking one or more of AR exons 3-7; or 4) a TRA with a breakpoint anywhere between the end of AR exon 3 and the stop codon in AR exon 8 that retains the centomeric side of AR encompassing exons 1-3.

Samples with AR-GSRs were classified as likely containing AR extrachromosomal circular DNA (AR ecDNA) if that sample contained at least 2 AR-GSRs and displayed copy number gain of the AR gene body, defined as log 2 (copy number ratio)>0.3.

Genome-Wide Copy Number Analysis with CNVkit

Genome wide copy number alterations were inferred using CNVkit software (ver. 0.9.9). An accessible_regions.bed input file was prepared using the CNVkit access function with two parameters: the GRCh37/hg19 reference genome, and the ENCODE project exclude BED file (wgEncodeDacMapabilityConsensusExcludable.bed). This file includes only sequence-accessible regions of the genome to be considered by CNVkit. The targets.bed input file was prepared using the CNVkit target function with three parameters: the targeted DNA-seq SureSelect probes BED file, the GRCh37/hg19 Ensembl GTF (release 87) for gene annotation, and the “split” parameter was specified (to divide larger baited regions into regions closer to the default bin size, 267 bp). The antitargets.bed input file was prepared using the CNVkit antitarget function with two parameters: the targets.bed and accessible_regions.bed files. For each sample, the CNVkit coverage function was used with the inputs described above and the deduplicated BAM file to calculate read coverage levels across the targeted and off-target regions of the genome. This step required the hmmlearn software (ver. 0.2.7). A CNVkit reference was built from the coverage files of 65 samples that had tumor fraction≤0.05, as determined by ichorCNA. The CNVkit reference function was performed with the “haploid-x-reference” and “sample-sex male” options to correctly estimate coverage levels in male samples. The CNVkit fix function was used to correct for known biases and calculate log 2 copy number ratios. The CNVkit segment function was completed using the “drop-low-coverage” option and the “cbs” segmentation algorithm. Finally, the CNVkit call function was used to calculate copy number integers from the log 2 ratios, with “haploid-x-reference”, “sample-sex male”, and “drop-low-coverage” parameters. For every sample, the inferred copy number ratios for each segment were plotted as a chromosomal heatmap using R and tidyverse functions. The log 2 ratio color scale was winsorized to the range: mean+/−5SD (to remove spurious outlier values skewing the signal).

Definition of ctDNA-Positive cfDNA Samples

The mean ctDNA aneuploidy fraction estimate of DNA-seq assays (n=8) performed on control cfDNA isolated from a pool of plasma from healthy male donors (ZenBio, Inc., Catalog #SER-PDP-10) was 0.0698 with a standard deviation of 0.0241. Samples exceeding the mean+3SD (>0.1421) were classified as ctDNA-positive and belonging to ctDNA aneuploidy-high Group 1 based on the rationale that samples below the mean+3SD cutoff are 99.87% likely to lack ctDNA aneuploidy. Accordingly, samples below this mean+3 SD cutoff were considered as ctDNA positive and belonging to ctDNA aneuploidy-low Group 2 only if they displayed at least one of the following features: a likely somatic SNV in any gene targeted by DNA-seq; AR log 2 (copy number ratio) of >0.3; AR enhancer log 2 (copy number ratio) of >0.3; positive for an AR-GSR; MYC log 2 (copy number ratio) of >0.3; MYCN log 2 (copy number ratio) of >0.3. Samples not classified in ctDNA aneuploidy-high Group 1 or ctDNA aneuploidy-low Group 2 were placed in ctDNA-negative Group 3.

### Statistical Analysis

A statistical analyses was performed to evaluate whether detection of AR-GSRs in ctDNA would be associated with worse overall survival (OS), and whether ctDNA positive patients would have adverse baseline clinical features, worse radiographic progression free survival (rPFS), and worse OS. cfDNA-sequencing and data analysis was performed to identify ctDNA-positive patient groups while blinded to clinical data. The comparisons were primarily focused on three groups: ctDNA-positive patients in ctDNA aneuploidy-high Group 1, ctDNA-positive patients in ctDNA aneuploidy-low Group 2, and ctDNA-negative Group 3. The analysis was focused to AR-GSRs as well as these ctDNA-positive and ctDNA-negative groups.

First, disease characteristics were compared between patients in ctDNA aneuploidy-high Group 1 and ctDNA aneuploidy-low Group 2, relative to those in ctDNA-negative Group 3. Specifically, the levels of prostate-specific antigen (PSA), hemoglobin, alkaline phosphatase, lactate dehydrogenase, as well as the frequencies of bone and liver metastasis were compared. This analysis aimed to determine any discernible differences in disease characteristics among the groups. The association was determined between the ctDNA classification and the risk factor, which is based on a prognostic model of OS. The Kaplan-Meier product-limit approach was used to estimate the rPFS and OS distributions by the different ctDNA classifications. The log-rank test was used to evaluate the statistical significance of differences between the two groups. The proportional hazards model was used to test for the prognostic significance in the groups predicting OS/rPFS adjusting for the ctDNA fraction. P-values are adjusted for multiplicity using the Benjamini-Hochberg method. The raw DNA-seq data for this study are available in dbGaP under accession: phs003325.v1.p1.

### Results

Baseline cfDNA Correlates with ctDNA Aneuploidy Fraction

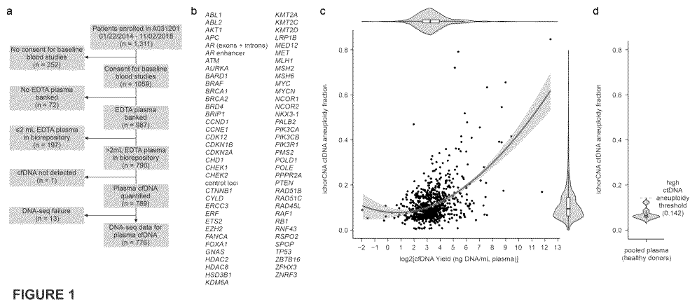

The Alliance A031201 trial was a randomized study of enzalutamide compared with enzalutamide plus abiraterone in first-line mCRPC patients. The treatment combination did not demonstrate a benefit in OS, although it demonstrated a modest 3 month delay on the secondary endpoint of rPFS. Blood specimens were collected and banked for all 1,311 patients evaluated in the trial, which were used for exploratory liquid biopsy analysis of plasma cell free DNA (cfDNA). Of the 1,059 patients that consented to baseline blood studies, there were 790 patients with at least 2 mL of plasma collected prior to treatment that was used for cfDNA isolation (FIG. 1a). Patients with less than or equal to 2 mL of plasma were excluded to avoid exhausting any patient plasma in the biorepository. A detectable level of cfDNA was isolated from 789/790 plasma specimens. These cfDNA specimens were analyzed using a research-grade paired-end DNA sequencing (DNA-seq) assay named AR-ctDETECT, which was designed to target 820,324 bp of genomic DNA representing control regions and 69 genes displaying recurrent alterations in mCRPC tumors (FIG. 1b).

Targeted DNA-seq was performed on 789 cfDNA samples, of which 776 passed quality control checks during library preparation and DNA-seq (FIG. 1a). These 776 patients had similar clinical baseline characteristics, treatment assignments, and outcomes to the 535 patients from the Alliance A031201 trial for which cfDNA-seq data were not available for analysis. Samples having very low cfDNA concentrations displayed higher percentages of DNA-seq reads attributed to PCR duplicates than those having high cfDNA concentrations. Accordingly, the cfDNA concentration of a sample was positively related to the mean unique read coverage of that sample after removal of PCR duplicates, which averaged 401X across all 776 samples. Comparable depths of coverage were achieved for all targeted genes.

The ichorCNA algorithm was developed to estimate circulating tumor DNA (ctDNA) fraction from aneuploidy detected in low-pass whole genome sequencing of cfDNA. Previous studies demonstrated the off-target reads from targeted DNA-seq assays can be used to approximate low-pass whole genome sequencing and be leveraged for estimating ctDNA fraction. Therefore, the on-target reads were removed from each sample and the remaining off-target reads used as input in ichorCNA. The median estimate from this approach was 9.4% ctDNA aneuploidy, with a range of 0.4% to 84.6% ctDNA aneuploidy (FIG. 1c). The ctDNA aneuploidy fraction was positively correlated with the yield of cfDNA from the corresponding plasma specimens (FIG. 1c). A test of the association of ctDNA aneuploidy fraction with clinical outcomes revealed that the hazard ratio (HR) for rPFS was 1.3 (95% CI: 1.2-1.4, P<0.0001) and the HR for OS was 1.5 (95% CI: 1.4-1.6, P<0.0001) for each 0.1 unit (10%) increase in ctDNA aneuploidy fraction.

Because ichorCNA can provide a ctDNA aneuploidy fraction estimate for any cfDNA sample, including those from healthy donors, the targeted DNA-seq assay described herein was used to analyze control plasma pooled from healthy donors. In these control plasma samples, ichorCNA provided estimates ranging from 4.7%-12.2% ctDNA aneuploidy (mean=7.0% and standard deviation=2.4%), which was used to derive an assay-specific threshold of 14.2% to distinguish cfDNA samples with high ctDNA aneuploidy (3 standard deviations beyond the control sample mean, FIG. 1d).

Baseline cfDNA Display a High Burden of AR Gene Alterations

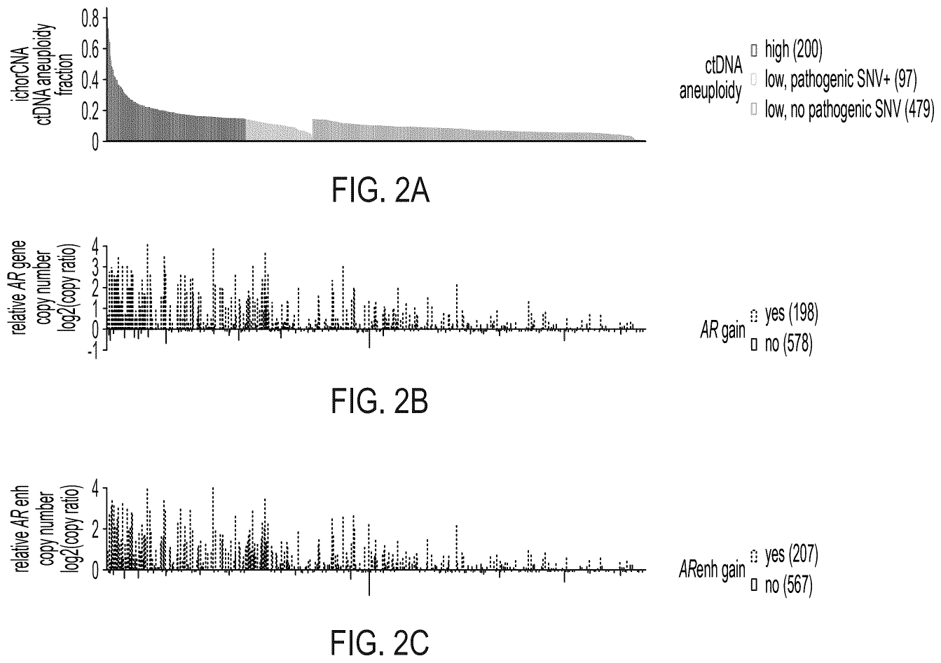

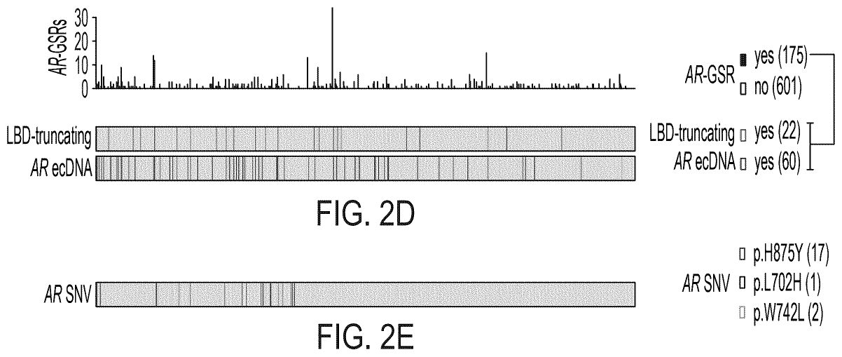

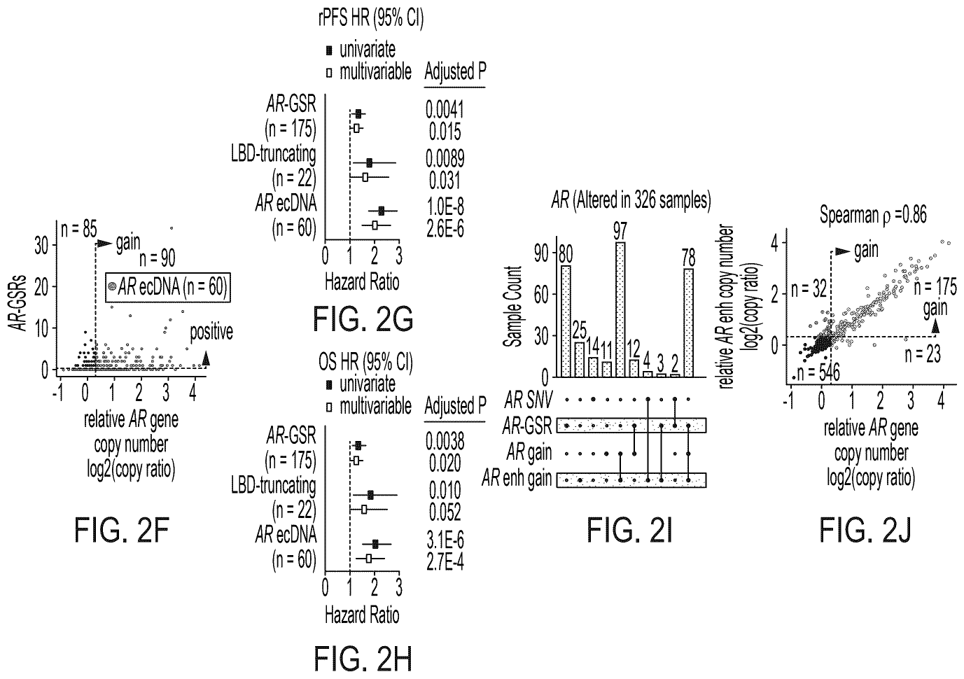

Based on this assay-specific ctDNA aneuploidy threshold, 26% of the plasma cfDNA specimens were classified as above the threshold as ctDNA aneuploidy-high (FIG. 2a). An additional method used to identify ctDNA in cfDNA is to leverage the detection of somatic mutations in genes targeted by DNA-seq. 309 mutations were identified that were pathogenic and classified as likely somatic. As expected, the majority of these pathogenic mutations occurred in ctDNA aneuploidy-high specimens. However, there were an additional 97 specimens below the ctDNA aneuploidy cutoff that harbored one or more of these pathogenic mutations, which was used to classify them as ctDNA-positive (FIG. 2a). It was noted that the patients with detected pathogenic mutations had a higher average yield of cfDNA isolated from their plasma specimens compared to those without detected pathogenic mutations. This finding may indicate a higher sensitivity for detecting ctDNA-specific mutations in plasma with greater cfDNA amounts, or higher levels of ctDNA in cfDNA with detected pathogenic mutations.

Few oncogenes and tumor suppressor genes are altered at high frequency by pathogenic mutations in mCRPC. Instead, mCRPC harbors a high degree of structural and copy number variation, and AR is frequently impacted by these events. Therefore, it was asked whether such AR alterations were detectable in cfDNA. The AR gene body displayed copy number gain in 198 cfDNA samples and the AR upstream enhancer displayed copy number gain in 207 cfDNA samples (FIGS. 2b, 2c). As expected, these AR copy gains were detected in many of the 200 ctDNA aneuploidy-high samples and the 97 ctDNA aneuploidy-low samples having a pathogenic SNV. However, these alterations were also apparent in many of the 479 cfDNA samples in the assay that would otherwise be classified as ctDNA-negative. Similarly, AR-GSRs, defined as structural variants with at least one breakpoint transecting the AR gene body, were detected in 175 cfDNA samples spanning those that were ctDNA-positive and otherwise ctDNA-negative (FIG. 2d). AR mutations (or single nucleotide variants, SNVs) were the least frequent AR alteration, occurring in 20 cfDNA samples (FIG. 2e). The most common AR mutation encoded a H875Y substitution in the AR protein.

AR-GSRs in Baseline cfDNA Associate with Poor Clinical Outcomes

Functionally, certain AR-GSRs have been shown to promote synthesis of truncated, constitutively active AR variant (AR-V) proteins lacking the AR ligand binding domain (LBD). There were a total of 457 AR-GSRs detected, with 57% of the AR-GSR-positive samples having 2 or more AR-GSRs. 54 AR-GSRs occurring in 22 cfDNA samples were classified as likely LBD-truncating events (FIG. 2d). 60 cfDNA samples with gain of the AR gene body and 2 or more AR-GSRs were classified as likely harboring AR ecDNA (FIGS. 2d, 2f). Noteworthy, cfDNA samples harboring AR-GSRs that were likely LBD-truncating and/or AR ecDNA-associated occurred in ctDNA-positive samples as well in cfDNA samples that were otherwise ctDNA-negative.

A prospective hypothesis of this study was that patients with AR-GSRs would have worse rPFS and OS. In univariate analysis and multivariable analysis corrected for ctDNA aneuploidy fraction, patients harboring AR-GSRs had worse rPFS and OS compared with patients that lacked AR-GSRs (FIGS. 2g, 2h). In univariate analysis, the hazard ratios for worse rPFS and OS were even higher in patients harboring AR-GSRs that were likely LBD-truncating or likely associated with AR ecDNA (FIGS. 2g, 2h). In multivariable analysis corrected for ctDNA aneuploidy fraction, these associations for AR-GSRs that were likely LBD-truncating or likely associated with AR ecDNA remained significant, with the exception of the association between likely LBD-truncating AR-GSRs and OS. Many of the samples with AR-GSRs were below the ctDNA aneuploidy fraction cutoff, which could impact the accuracy of using ctDNA aneuploidy fraction for multivariable correction. Therefore, the association of ctDNA aneuploidy fraction was tested as a continuous variable with clinical outcomes in the 479 patients with low ctDNA aneuploidy and lacking a detectable pathogenic mutation. Remarkably, even in this subgroup, ctDNA aneuploidy fraction remained prognostic for rPFS (HR=1.05 for each 1% increase in ctDNA aneuploidy fraction, 95% CI: 1.01-1.09) and OS (HR=1.05 for each 1% increase in ctDNA aneuploidy fraction, 95% CI: 1.01-1.09).

AR Alterations and MYC/MYCN Gains Identify ctDNA-Positive Patients

Collectively, 326 of all cfDNA samples analyzed by DNA-seq displayed at least one AR gene alteration (FIG. 2i). The most frequent co-occurring AR gene alterations were copy number gains of the AR gene body and upstream enhancer. Only 23 of the cfDNA samples with AR gene body gain lacked an accompanying AR upstream enhancer gain, and 32 of the cfDNA samples with AR upstream enhancer gain lacked an accompanying AR gene body gain (FIGS. 2i, 2j). Indeed, there was very high correlation in the relative copy number of the AR gene body and upstream enhancer, which is consistent with the notion that these two genomic regions are usually contained on the same amplicon in mCRPC tissues (FIG. 2j)

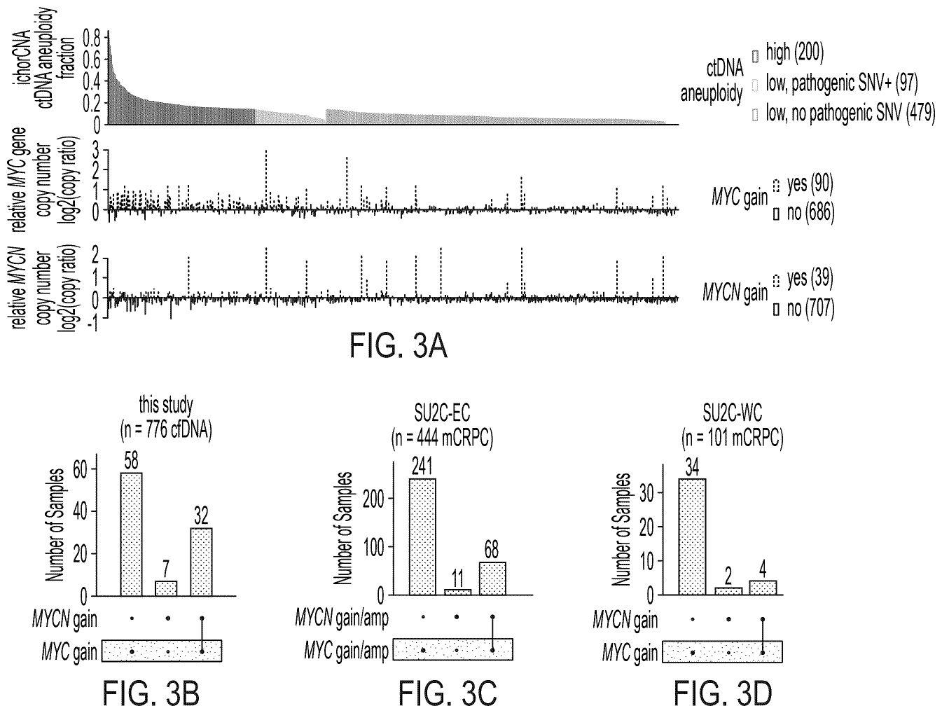

Because mCRPC-specific AR amplification was apparent in cfDNA that might otherwise be classified as ctDNA-negative, it was assessed whether additional amplification events could be leveraged for their high ctDNA-specific signal. Amplification of MYC and MYCN are somatic events that occur with high frequency in mCRPC genomes. MYC displayed copy number gain in 90 cfDNA samples and MYCN gain displayed copy number gain in 39 cfDNA samples (FIG. 3a). Although most of these MYC and/or MYCN copy number gains occurred in ctDNA-positive samples, they were also detectable in cfDNA samples that were otherwise cfDNA-negative (FIG. 3a). Notably, 82% of the cfDNA samples that displayed MYCN gain also displayed MYC gain (FIG. 3b). Concurrent gain of MYC and MYCN has not been reported in mCRPC previously. To confirm this was not an artifact of the AR-ctDETECT assay or analysis of cfDNA, MYC and MYCN gain was evaluated in two public datasets of whole exome or whole genome DNA-seq analysis of mCRPC tissue biopsies. Both of these datasets confirmed this co-occurrence, with 86% or 67% of the mCRPC tissues with MYCN gains also having MYC gains (FIGS. 3c, 3d).

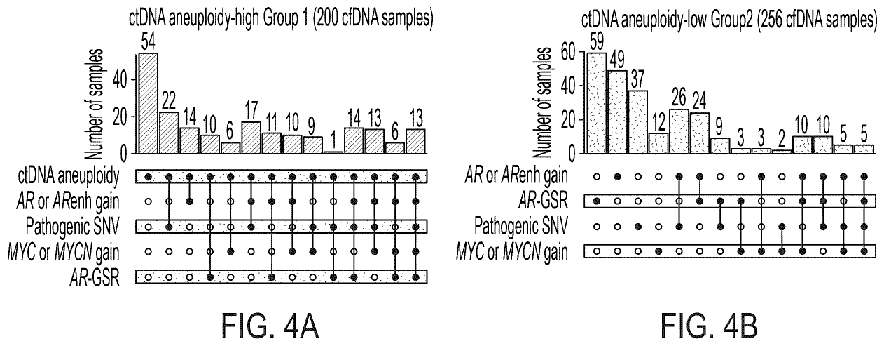

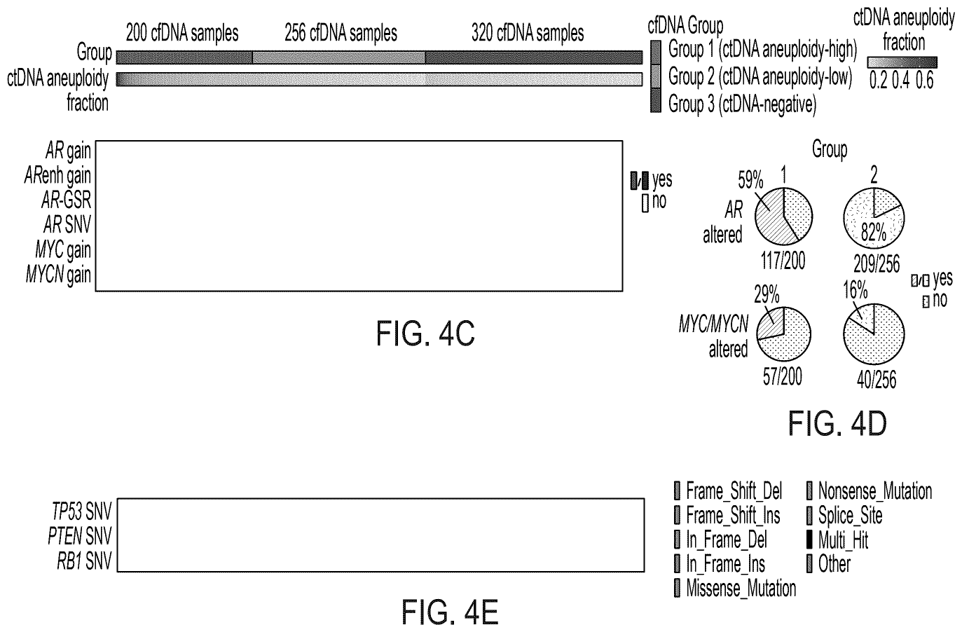

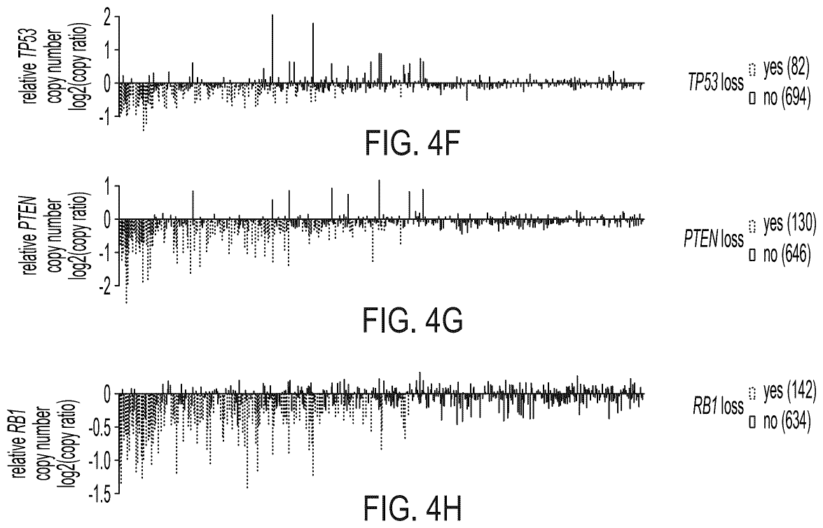

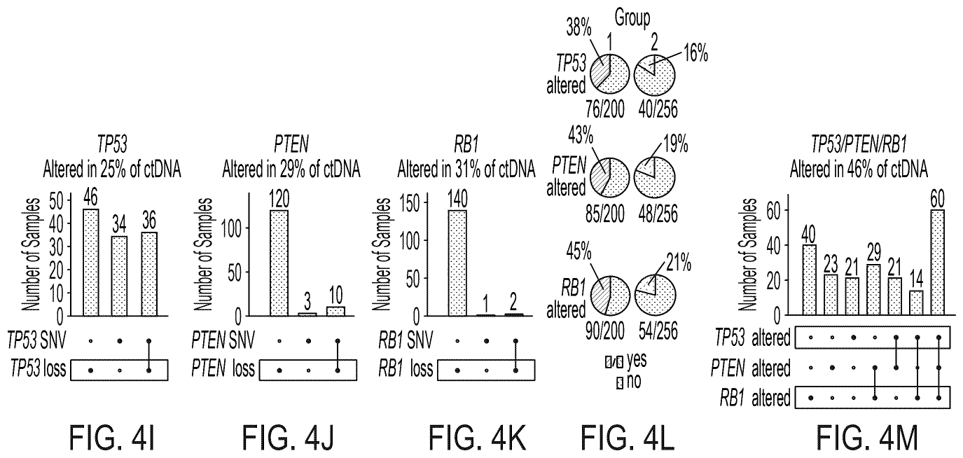

Based on the observations that alterations of AR, MYC, or MYCN were detectable in cfDNA samples that would otherwise be classified as ctDNA-negative by aneuploidy or mutation-based methods, the clinical significance of incorporating AR, MYC or MYCN alterations into ctDNA detection was evaluated. Across the 479 patients that were ctDNA aneuploidy-low and lacked a pathogenic mutation, OS and rPFS were longer in the 320 patients that lacked detectable alterations in AR, MYC or MYCN compared with the 159 patients where these alterations were detected. Based on these supporting results, 776 cfDNA samples were stratified into 3 groups. The first group of 200 cfDNA samples were those originally classified by ichorCNA as ctDNA aneuploidy-high (FIG. 4a). The second group of 256 cfDNA samples were those classified by ichorCNA as ctDNA aneuploidy-low, but deemed to be ctDNA-positive on the basis of harboring one or more likely somatic pathogenic mutations in any targeted gene and/or detectable alterations in AR, MYC or MYCN (FIGS. 4b, 4c). Overall, 59% of all cfDNA samples were ctDNA positive based on these classifications (FIG. 4c). AR alterations occurred in 71% of ctDNA-positive specimens, with the highest frequency in ctDNA aneuploidy-low Group 2 (FIG. 4d). In contrast, MYC and/or MYCN alterations occurred in 21% of ctDNA-positive specimens, with the highest frequency in ctDNA aneuploidy-high Group 1 (FIG. 4d).

Alterations in Common Prostate Cancer Genes are Frequently Detected in ctDNA-High Patients

Mutations in TP53, PTEN, and RB1 in ctDNA aneuploidy-high Group 1 and ctDNA aneuploidy-low Group 2 were investigated. TP53 displayed the highest mutational frequency among all the genes targeted for DNA-seq analysis, with 15.3% of the ctDNA-positive samples harboring a TP53 mutation (FIG. 4e). The mutational frequencies of PTEN (2.9% of ctDNA-positive samples) and RB1 (0.7% of ctDNA-positive samples) were much lower.

In addition to mutations, substantial copy number loss was detectable for TP53, PTEN, and RB1 in ctDNA-positive samples (FIGS. 4f-4h). By aggregating the data for mutations and copy number losses, it was observed that TP53 was altered in 25% of the ctDNA-positive samples (FIG. 4i), PTEN was altered in 29% of the ctDNA-positive samples (FIG. 4j), and RB1 was altered in 31% of the ctDNA-positive samples (FIG. 4k). Comparing the proportions of samples belonging to ctDNA aneuploidy-high Group 1 or ctDNA aneuploidy-low Group 2 that harbored detectable mutations and/or copy number loss in TP53, PTEN, or RB1 revealed 2.1-2.4 fold higher rates of alterations in these genes in samples belonging to ctDNA aneuploidy-high Group 1 (FIG. 4l). Overall, 46% of the ctDNA-positive samples displayed at least 1 alteration in TP53, PTEN, or RB1 (FIG. 4m). Further, 13% of the ctDNA-positive samples displayed alterations in all 3 tumor suppressor genes, with all but 6 of these 55 samples belonging to ctDNA-high Group 1.

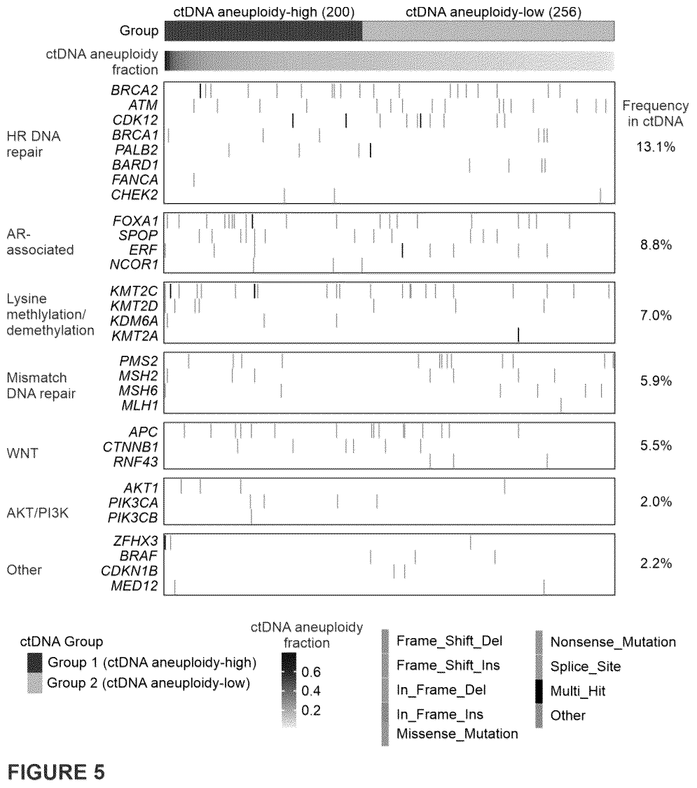

Extending the analysis to include all genes targeted by DNA-seq revealed an additional 30 genes that harbored detectable pathogenic mutations in ctDNA-positive samples (FIG. 5). Because the mutational frequency of any individual gene was low, these were grouped functionally to assess their prevalence among ctDNA-positive samples. The most frequently-altered pathways were those associated with homologous recombination DNA repair and AR regulation. The mutation frequency for genes in these and other pathways were similar between ctDNA aneuploidy-high Group 1 samples and ctDNA aneuploidy-low Group 2 samples.

The genetic alterations detected in ctDNA were used to benchmark the ctDNA aneuploidy fraction estimates derived from using off-target reads as input for ichorCNA. For instance, the variant allele fraction (VAF) of somatic tumor-derived mutations has been used for calculating ctDNA fraction in studies where high plasma volumes yielded high cfDNA concentrations, enabling high sequencing depth and sensitive mutation detection. The VAFs of mutations detected across 183 cfDNA specimens were higher in samples having high ctDNA aneuploidy than in samples having low ctDNA aneuploidy. Further, the maximum VAF detected in each of these 183 cfDNA specimens displayed a positive correlation with the ctDNA aneuploidy fraction. This positive correlation between maximum VAF and ctDNA aneuploidy fraction was highest for TP53, which was the most frequently-mutated gene in this study. Another input that has been used to calculate ctDNA fraction is the magnitude of tumor-derived somatic copy number gains or losses of targeted genes. Positive correlations were observed between the ctDNA aneuploidy fraction and magnitude of copy number gain detected in the AR gene body or enhancer in cfDNA samples where these gains occurred, and a negative correlation between the ctDNA aneuploidy fraction and magnitude of copy number loss in TP53, PTEN, or RB1 in cfDNA samples where these losses occurred. Finally, CNVkit is an algorithm to infer and visualize tumor-derived copy number alterations from targeted DNA-seq data. Visual inspection of the CNVkit output for all 776 plasma specimens confirmed a high degree of genome-wide copy number gains and losses in samples predicted by ichorCNA to have high ctDNA aneuploidy, and a relative paucity of genome-wide copy number gains and losses in samples below the ichorCNA ctDNA aneuploidy threshold. Collectively, these benchmarking data demonstrate that the ctDNA aneuploidy fraction estimated by ichorCNA correlates with inputs used to calculate ctDNA fraction in cfDNA.

ctDNA-Positive Patients have Poor Clinical Outcomes

A prospective hypothesis of this study was that ctDNA-positive patients would have poor prognosis. mCRPC patients in ctDNA aneuploidy-high Group 1 had the highest average plasma cfDNA concentration, patients in ctDNA-negative Group 3 had the lowest average plasma cfDNA concentration, and patients in ctDNA aneuploidy-low Group 2 had an intermediate average plasma cfDNA concentration. Relationships with prognostic risk at baseline were next investigated. Compared to patients in ctDNA-negative Group 3, patients in ctDNA aneuploidy-high Group 1 or ctDNA aneuploidy-low Group 2 were more likely to be classified as high risk and less likely to be classified as low risk, and had higher Halabi Risk scores, which are based on a multivariate prognostic model of OS.

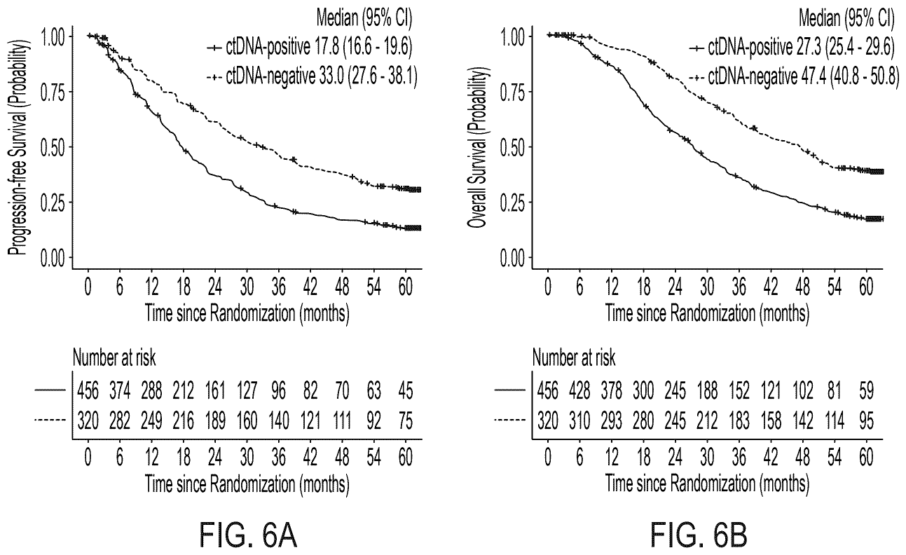

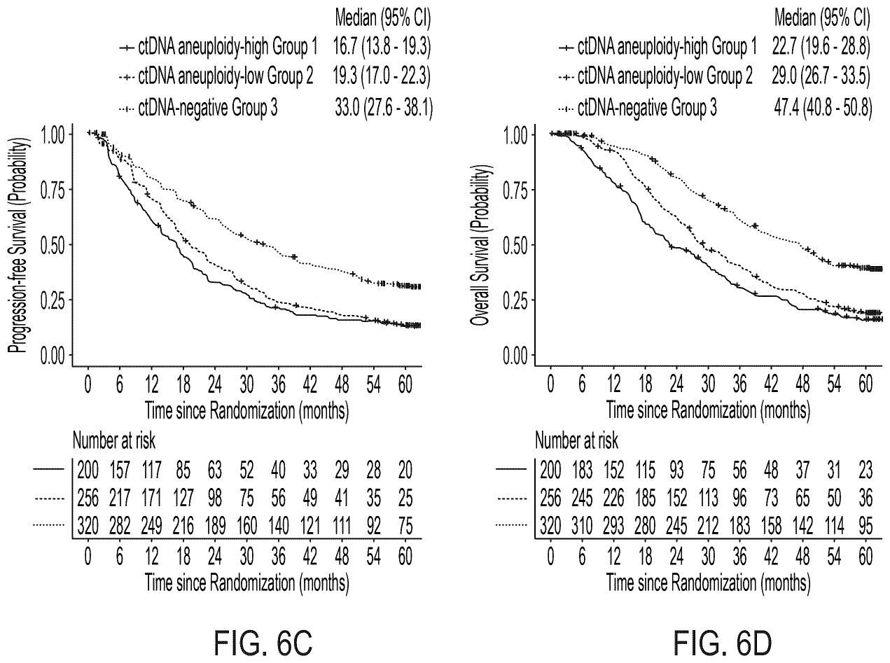

To further test this prospective hypothesis, survival analysis was performed. Patients negative for ctDNA had median rPFS of 33.0 months (95% CI 27.6-38.1 months) and median OS of 47.4 months (95% CI 40.8-50.8 months). In contrast, ctDNA-positive patients had shorter median rPFS of 17.8 months (95% confidence interval 16.6-19.6 months, FIG. 6a) and shorter median OS of 27.3 months (95% confidence interval 25.4-29.6 months, FIG. 6b). The HR for rPFS was 1.8 (95% CI: 1.5-2.1, P<0.0001) and the HR for OS was 2.0 (95% CI: 1.7-2.4, P<0.0001) in ctDNA-positive vs. ctDNA-negative patients.

When ctDNA-positive patients were stratified into ctDNA aneuploidy-high Group 1 and ctDNA-aneuploidy-low Group 2, patients in Group 1 had median rPFS of 16.7 months (95% CI 13.8-19.3 months, FIG. 6c). The hazard ratio (HR) for rPFS was 1.9 (95% CI: 1.6-2.4, P<0.0001) in Group 1 compared with Group 3. Patients in Group 2 had a median rPFS of 19.3 months (95% confidence interval 17.0-22.3 months). The HR for rPFS was 1.7 (95% CI: 1.4-2.3, P<0.0001) for Group 2 compared with Group 3. There was no statistical difference in rPFS between Groups 1 and 2 (HR=1.1, 95% CI: 0.9-1.4, P=0.21).

Patients in ctDNA aneuploidy-high Group 1 had the shortest OS duration with a median of 22.7 months (95% CI 19.6-28.8 months, FIG. 6d). The HR for OS was 2.3 (95% CI: 1.8-2.8, P<0.0001) in Group 1 compared with ctDNA-negative Group 3. In contrast, patients in ctDNA aneuploidy-low Group 2 had a median OS of 29.0 months (95% CI 26.7-33.5 months, FIG. 6d). The HR for OS was 1.8 (95% CI: 1.5-2.2, P<0.0001) in Group 2 compared with ctDNA-negative Group 3. The HR for OS was higher in Group 1 compared with Group 2 (HR=1.3, 95% CI: 1.0-1.6, P=0.03).

When the patients in ctDNA aneuploidy-low Group 2 were analyzed as two subgroups, the 97 patients in Group 2 that harbored detectable pathogenic mutations had worse rPFS and OS than patients in ctDNA-negative Group 3, but rPFS and OS that were indistinguishable from ctDNA aneuploidy-high Group 1. The 159 patients in Group 2 that lacked detectable pathogenic mutations but harbored AR-GSRs and/or a copy gain in AR, MYC, or MYCN had worse rPFS and OS than patients in ctDNA-negative Group 3, and better rPFS (but not OS) than patients in ctDNA aneuploidy-high Group 1. Collectively, these data demonstrate clinical utility of leveraging AR-GSRs and/or a copy gain in AR, MYC, or MYCN along with more traditional use of somatic mutations for detecting ctDNA in patients with low/negative ctDNA aneuploidy. Overall, patients classified as ctDNA-positive by the AR-ctDETECT cfDNA sequencing assay have poor clinical outcomes despite treatment with potent AR inhibition in a phase 3 trial.

### DISCUSSION

As described herein, a new and useful assay was developed to analyze cfDNA specimens from men with newly-diagnosed mCRPC enrolled in the randomized phase 3 Alliance A031201 trial testing enzalutamide vs. enzalutamide plus abiraterone. 776 cfDNA specimens for which at least 2 mL plasma was available were analyzed, and patients were stratified into 3 groups based on distinguishing characteristics in their cfDNA. There were 200 patients in ctDNA aneuploidy-high Group 1, which were ctDNA-positive based on a definition of exceeding an assay-specific ctDNA aneuploidy fraction greater than 14.2% estimated by the ichorCNA algorithm. However, using a strategy of defining as ctDNA-positive those patients harboring a known pathogenic mutation, an AR-GSR, and/or a copy gain in AR, MYC or MYCN, we identified 256 patients in a ctDNA aneuploidy-low Group 2 that had a poor prognosis despite having an estimated ctDNA aneuploidy fraction at or below the assay-specific threshold and receiving potent AR inhibition. Over 80% of the patients in ctDNA aneuploidy-low Group 2 had at least 1 AR alteration. These molecular alterations have a high signal to noise ratio in cfDNA given their high level of gain, and thus detection ability is enhanced as compared to copy losses or lower level gains. Overall survival was shorter in patients belonging to ctDNA aneuploidy-high Group 1 and ctDNA aneuploidy-low Group 2 compared with ctDNA-negative Group 3, which is an important finding. This indicates that the ctDNA aneuploidy-low Group 2, which is mainly defined by AR alterations, has a poor prognosis despite having a low level of ctDNA aneuploidy. Interesting, the patients in ctDNA aneuploidy-high Group 1 had worse OS (but not rPFS) compared to patients in ctDNA aneuploidy-low Group 2. Collectively, these clinical associations support this unique strategy of leveraging the high-signal copy number gains in AR, MYC, and MYCN, as well as AR-GSRs and combining them with the more traditional use of detectable pathogenic mutations to classify otherwise ctDNA-low/negative cfDNA samples as being ctDNA-positive. Further, it is believed that this is the first demonstration in a phase 3 cohort that ctDNA-positive mCRPC patients treated with first-line potent AR-targeted therapies had significantly worse OS compared to ctDNA-negative mCRPC patients.

When ctDNA-positive patients in ctDNA aneuploidy-high Group 1 and ctDNA aneuploidy-low Group 2 were combined, it was found they had a median OS of 27.3 months (95% confidence interval, 25.4-29.6 months), whereas ctDNA-negative patients in Group 3 had a median OS of 47.4 months (95% confidence interval, 40.8-50.8 months). Adverse clinical factors have also been used to stratify all 1,311 patients in the A031201 trial into Halabi Risk Factor groups, with a 3-tier prognostic model of high, intermediate, and low risk groups stratifying patients by OS. Associations between ctDNA status and these Halabi Risk Factor groups were noted, including a higher frequency of the high-risk classification among patients positive for ctDNA, and a higher frequency of the low-risk classification among patients negative for ctDNA. However, there were many patients in the low- and intermediate-risk groups that were in ctDNA aneuploidy-high Group 1 and ctDNA aneuploidy-low Group 2, indicating a potential for success with our planned future study to combine adverse clinical factors and ctDNA detection into a unified clinical-genomic model for improving patient prognostication.

This study also highlights a potential utility of using targeted DNA-seq of cfDNA to identify key alterations in mCRPC genomes. Among the 456 ctDNA-positive cfDNA samples analyzed in this study, AR was altered in 326 samples, nominating it as the most frequently-altered gene in this context. The three most common AR alterations were copy gain of the AR upstream enhancer, copy gain of the AR gene body, and AR-GSRs, which alter the structure of the AR gene. The frequent co-occurrence of these AR alterations indicates that a substantial proportion of mCRPC is characterized by complex patterns of amplified and rearranged AR gene structures, which in certain cases can be explained by AR ecDNA. The majority of the cfDNA samples in ctDNA aneuploidy-low Group 2 were classified as ctDNA-positive as a result of displaying these complex patterns of AR alterations. The fact that patients belonging to ctDNA aneuploidy-low Group 2 demonstrated a worse OS relative to ctDNA-negative Group 3 is consistent with the notion that these AR alterations drive broad resistance to AR-targeted therapies, including potent drugs like enzalutamide and/or abiraterone. It should be noted that all patients in the phase 3 A031201 trial were treated with enzalutamide or enzalutamide plus abiraterone, so it will be important to determine whether patients in ctDNA aneuploidy-low Group 2 also have worse OS in trials of drugs that are independent of AR signaling.

The 200 cfDNA samples in ctDNA aneuploidy-high Group 1 harbored the majority of the non-AR genomic alterations that were detected by the AR-ctDETECT assay. This included frequent mutations and/or copy number losses in tumor suppressors TP53, PTEN, and RB1, and copy number gains in MYC and/or MYCN. Conversely, samples in ctDNA aneuploidy-low Group 2 displayed lower rates of these alterations, which is likely due to the lower sensitivity for detecting mutations and/or copy number losses in samples with low ctDNA content. Many of the alterations in these genes promote mCRPC resistance to endocrine therapies and are negative prognostic diagnostics when detected in tumor tissue and/or cfDNA. Therefore, there may be utility of incorporating specific gene alterations into prognostic models that include clinical factors and ctDNA fractions. This is further supported by the finding that AR-GSRs, especially those that are predicted to truncate the AR LBD and/or arise through AR ecDNA amplification, are associated with increased risk of radiographic progression and death.

It is noted that out of the 1,311 patients treated in the phase 3 A031201 trial, plasma specimens were only available from 987 of these patients and this study analyzed 790 of them. This is because analysis of the remaining 197 plasma specimens would have exhausted them in the biorepository. Additionally, the low volumes of plasma available for analysis led to low yields of cfDNA in many samples, reducing the sensitivity for detecting ctDNA-specific alterations. These technical limitations likely contributed to false negatives among the patients categorized as ctDNA-negative. A final limitation was that targeted DNA-seq was performed on cfDNA without patient-matched germline controls, blood, or tumor tissue. This limits the ability to accurately distinguish between somatic or germline alterations in target genes, or between tumor-associated mutations and clonal hematopoiesis. To address this, only high confidence mutations with well-annotated pathogenicity were considered (from OncoKB). This may have resulted in under-reporting of mutations in some genes targeted by the AR-ctDETECT assay and also restricts the ability to determine tumor mutational burden. Accurately detecting tumor-associated mutations in cfDNA and measuring their corresponding VAFs is important for determining ctDNA fractions in cfDNA. Due to the lower sensitivity for detecting these mutations in this study, a ctDNA aneuploidy fraction for all samples was calculated using the ichorCNA algorithm. Although this ctDNA aneuploidy fraction calculation was prognostic for rPFS and OS, and correlated with other inputs used to calculate ctDNA fraction, these metrics are distinct and should not be directly compared.

In summary, analysis of this phase 3 study has demonstrated the prognostic utility of a mCRPC-specific cfDNA-seq assay that leverages detection of tumor-derived copy gain and structural alterations in AR, as well as copy gain in MYC and/or MYCN. Detection of ctDNA in general and specific genomic alterations in particular will improve on established clinical factors for developing composite prognostic diagnostics.

### CITATIONS

1. Garraway, L. A. & Sellers, W. R. Lineage dependency and lineage-survival oncogenes in human cancer. Nat Rev Cancer 6, 593-602 (2006).
2. Watson, P. A., Arora, V. K. & Sawyers, C. L. Emerging mechanisms of resistance to androgen receptor inhibitors in prostate cancer. Nat Rev Cancer 15, 701-711 (2015).
3. Scher, H. I., et al. Trial Design and Objectives for Castration-Resistant Prostate Cancer: Updated Recommendations From the Prostate Cancer Clinical Trials Working Group 3. J Clin Oncol 34, 1402-1418 (2016).
4. Ryan, C. J., et al. Abiraterone in metastatic prostate cancer without previous chemotherapy. N Engl J Med 368, 138-148 (2013).
5 Beer, T. M., et al. Enzalutamide in metastatic prostate cancer before chemotherapy. N Engl J Med 371, 424-433 (2014).
6. Scher, H. I., et al. Association of AR-V7 on Circulating Tumor Cells as a Treatment-Specific Biomarker With Outcomes and Survival in Castration-Resistant Prostate Cancer. JAMA Oncol 2, 1441-1449 (2016).
7. Antonarakis, E. S., et al. Androgen Receptor Splice Variant 7 and Efficacy of Taxane Chemotherapy in Patients With Metastatic Castration-Resistant Prostate Cancer. JAMA oncology 1, 582-591 (2015).
8. Parker, C., et al. Alpha emitter radium-223 and survival in metastatic prostate cancer. The New England journal of medicine 369, 213-223 (2013).
9. Tannock, I. F., et al. Docetaxel plus prednisone or mitoxantrone plus prednisone for advanced prostate cancer. N Engl J Med 351, 1502-1512 (2004).
10. Hussain, M., et al. Survival with Olaparib in Metastatic Castration-Resistant Prostate Cancer. N Engl J Med 383, 2345-2357 (2020).
11. Mateo, J., et al. DNA-Repair Defects and Olaparib in Metastatic Prostate Cancer. N Engl J Med 373, 1697-1708 (2015).
12. Fizazi, K., et al. Rucaparib or Physician's Choice in Metastatic Prostate Cancer. N Engl J Med 388, 719-732 (2023).
13. Marabelle, A., et al. Efficacy of Pembrolizumab in Patients With Noncolorectal High Microsatellite Instability/Mismatch Repair-Deficient Cancer: Results From the Phase II KEYNOTE-158 Study. J Clin Oncol 38, 1-10 (2020).
14. Halabi, S., et al. External Validation of a Prognostic Model of Overall Survival in Men With Chemotherapy-Naive Metastatic Castration-Resistant Prostate Cancer. J Clin Oncol 41, 2736-2746 (2023).
15. Armstrong, A. J., et al. Five-year Survival Prediction and Safety Outcomes with Enzalutamide in Men with Chemotherapy-naive Metastatic Castration-resistant Prostate Cancer from the PREVAIL Trial. Eur Urol 78, 347-357 (2020).
16. Mehra, N., et al. Plasma Cell-free DNA Concentration and Outcomes from Taxane Therapy in Metastatic Castration-resistant Prostate Cancer from Two Phase III Trials (FIRSTANA and PROSELICA). Eur Urol 74, 283-291 (2018).
17. Annala, M., et al. Circulating Tumor DNA Genomics Correlate with Resistance to Abiraterone and Enzalutamide in Prostate Cancer. Cancer discovery 8, 444-457 (2018).
18. De Laere, B., et al. TP53 Outperforms Other Androgen Receptor Biomarkers to Predict Abiraterone or Enzalutamide Outcome in Metastatic Castration-Resistant Prostate Cancer. Clin Cancer Res 25, 1766-1773 (2019).
19. Annala, M., et al. Cabazitaxel versus abiraterone or enzalutamide in poor prognosis metastatic castration-resistant prostate cancer: a multicentre, randomised, open-label, phase II trial. Annals of oncology: official journal of the European Society for Medical Oncology/ESMO 32, 896-905 (2021).
20. Choudhury, A. D., et al. Tumor fraction in cell-free DNA as a biomarker in prostate cancer. JCI Insight 3 (2018).
21. Carreira, S., et al. Tumor clone dynamics in lethal prostate cancer. Science translational medicine 6, 254ra125 (2014).
22. Annala, M., et al. Evolution of Castration-Resistant Prostate Cancer in ctDNA during Sequential Androgen Receptor Pathway Inhibition. Clin Cancer Res 27, 4610-4623 (2021).
23. Azad, A. A., et al. Androgen Receptor Gene Aberrations in Circulating Cell-Free DNA: Biomarkers of Therapeutic Resistance in Castration-Resistant Prostate Cancer. Clin Cancer Res 21, 2315-2324 (2015).
24. Conteduca, V., et al. Androgen receptor gene status in plasma DNA associates with worse outcome on enzalutamide or abiraterone for castration-resistant prostate cancer: a multi-institution correlative biomarker study. Annals of oncology: official journal of the European Society for Medical Oncology/ESMO 28, 1508-1516 (2017).
25. Romanel, A., et al. Plasma AR and abiraterone-resistant prostate cancer. Science translational medicine 7, 312re310 (2015).
26. Wyatt, A. W., et al. Genomic Alterations in Cell-Free DNA and Enzalutamide Resistance in Castration-Resistant Prostate Cancer. JAMA oncology 2, 1598-1606 (2016).
27. Swami, U., et al. SPOP Mutations as a Predictive Biomarker for Androgen Receptor Axis-Targeted Therapy in De Novo Metastatic Castration-Sensitive Prostate Cancer. Clin Cancer Res 28, 4917-4925 (2022).
28. Tukachinsky, H., et al. Genomic Analysis of Circulating Tumor DNA in 3,334 Patients with Advanced Prostate Cancer Identifies Targetable BRCA Alterations and AR Resistance Mechanisms. Clin Cancer Res (2021).
29. Reichert, Z. R., et al. Prognostic value of plasma circulating tumor DNA fraction across four common cancer types: a real-world outcomes study. Annals of oncology: official journal of the European Society for Medical Oncology/ESMO 34, 111-120 (2023).
30. Kohli, M., et al. Clinical and genomic insights into circulating tumor DNA-based alterations across the spectrum of metastatic hormone-sensitive and castrate-resistant prostate cancer. EBioMedicine 54, 102728 (2020).
31. Morris, M. J., et al. Randomized Phase III Study of Enzalutamide Compared With Enzalutamide Plus Abiraterone for Metastatic Castration-Resistant Prostate Cancer (Alliance A031201 Trial). J Clin Oncol, JCO2202394 (2023).
32. Adalsteinsson, V. A., et al. Scalable whole-exome sequencing of cell-free DNA reveals high concordance with metastatic tumors. Nature communications 8, 1324 (2017).
33. Finkle, J. D., et al. Validation of a liquid biopsy assay with molecular and clinical profiling of circulating tumor DNA. NPJ Precis Oncol 5, 63 (2021).
34. Shah, A. T., et al. A Comprehensive Circulating Tumor DNA Assay for Detection of Translocation and Copy-Number Changes in Pediatric Sarcomas. Mol Cancer Ther 20, 2016-2025 (2021).
35. De Laere, B., et al. Comprehensive Profiling of the Androgen Receptor in Liquid Biopsies from Castration-resistant Prostate Cancer Reveals Novel Intra-AR Structural Variation and Splice Variant Expression Patterns. Eur Urol (2017).
36. Orlando, F., et al. Allele-informed copy number evaluation of plasma DNA samples from metastatic prostate cancer patients: the PCF_SELECT consortium assay. NAR Cancer 4, zcac016 (2022).
37. Armenia, J., et al. The long tail of oncogenic drivers in prostate cancer. Nat Genet 50, 645-651 (2018).
38. Quigley, D. A., et al. Genomic Hallmarks and Structural Variation in Metastatic Prostate Cancer. Cell 174, 758-769 e759 (2018).
39. Robinson, D., et al. Integrative clinical genomics of advanced prostate cancer. Cell 161, 1215-1228 (2015).
40. Zivanovic, A., et al. Co-evolution of AR gene copy number and structural complexity in endocrine therapy resistant prostate cancer. NAR Cancer 5, zcad045 (2023).
41. Martinez-Jimenez, F., et al. Pan-cancer whole-genome comparison of primary and metastatic solid tumours. Nature 618, 333-341 (2023).
42. Li, Y., et al. Diverse AR Gene Rearrangements Mediate Resistance to Androgen Receptor Inhibitors in Metastatic Prostate Cancer. Clin Cancer Res (2020).
43. Lawrence, M. G., et al. Patient-derived Models of Abiraterone- and Enzalutamide-resistant Prostate Cancer Reveal Sensitivity to Ribosome-directed Therapy. Eur Urol 74, 562-572 (2018).
44. Henzler, C., et al. Truncation and constitutive activation of the androgen receptor by diverse genomic rearrangements in prostate cancer. Nature communications 7, 13668 (2016).
45. Nyquist, M. D., et al. TALEN-engineered AR gene rearrangements reveal endocrine uncoupling of androgen receptor in prostate cancer. Proc Natl Acad Sci USA 110, 17492-17497 (2013).
46. Li, Y., et al. AR intragenic deletions linked to androgen receptor splice variant expression and activity in models of prostate cancer progression. Oncogene 31, 4759-4767 (2012).
47. Li, Y., et al. Intragenic rearrangement and altered RNA splicing of the androgen receptor in a cell-based model of prostate cancer progression. Cancer Res 71, 2108-2117 (2011).
48. Takeda, D. Y., et al. A Somatically Acquired Enhancer of the Androgen Receptor Is a Noncoding Driver in Advanced Prostate Cancer. Cell 174, 422-432 e413 (2018).
49. Viswanathan, S. R., et al. Structural Alterations Driving Castration-Resistant Prostate Cancer Revealed by Linked-Read Genome Sequencing. Cell 174, 433-447 e419 (2018).
50. Beltran, H., et al. Molecular characterization of neuroendocrine prostate cancer and identification of new drug targets. Cancer discovery 1, 487-495 (2011).
51. Mosquera, J. M., et al. Concurrent AURKA and MYCN gene amplifications are harbingers of lethal treatment-related neuroendocrine prostate cancer. Neoplasia 15, 1-10 (2013).
52. Abida, W., et al. Genomic correlates of clinical outcome in advanced prostate cancer. Proc Natl Acad Sci USA 116, 11428-11436 (2019).
53. Talevich, E., Shain, A. H., Botton, T. & Bastian, B. C. CNVkit: Genome-Wide Copy Number Detection and Visualization from Targeted DNA Sequencing. PLOS Comput Biol 12, e1004873 (2016).
54. Kelly, W. K., et al. Randomized, double-blind, placebo-controlled phase III trial comparing docetaxel and prednisone with or without bevacizumab in men with metastatic castration-resistant prostate cancer: CALGB 90401. J Clin Oncol 30, 1534-1540 (2012).
55. Jayaram, A., et al. Plasma tumor gene conversions after one cycle abiraterone acetate for metastatic castration-resistant prostate cancer: a biomarker analysis of a multicenter international trial. Annals of oncology: official journal of the European Society for Medical Oncology/ESMO 32, 726-735 (2021).
56. Zhou, M., et al. Patterns of structural variation define prostate cancer across disease states. JCI Insight 7 (2022).
57. Chakravarty, D., et al. OncoKB: A Precision Oncology Knowledge Base. JCO Precis Oncol 2017 (2017).
58. Bolger, A. M., Lohse, M. & Usadel, B. Trimmomatic: a flexible trimmer for Illumina sequence data. Bioinformatics 30, 2114-2120 (2014).
59. Smith, T., Heger, A. & Sudbery, I. UMI-tools: modeling sequencing errors in Unique Molecular Identifiers to improve quantification accuracy. Genome Res 27, 491-499 (2017).
60. Cibulskis, K., et al. Sensitive detection of somatic point mutations in impure and heterogeneous cancer samples. Nat Biotechnol 31, 213-219 (2013).
61. Karczewski, K. J., et al. The mutational constraint spectrum quantified from variation in 141,456 humans. Nature 581, 434-443 (2020).
62. Tan, A., Abecasis, G. R. & Kang, H. M. Unified representation of genetic variants. Bioinformatics 31, 2202-2204 (2015).
63. McLaren, W., et al. The Ensembl Variant Effect Predictor. Genome Biol 17, 122 (2016).
64. Pedersen, B. S. & Quinlan, A. R. Mosdepth: quick coverage calculation for genomes and exomes. Bioinformatics 34, 867-868 (2018).
65. Chen, X., et al. Manta: rapid detection of structural variants and indels for germline and cancer sequencing applications. Bioinformatics 32, 1220-1222 (2016).
66. Rausch, T., et al. DELLY: structural variant discovery by integrated paired-end and split-read analysis. Bioinformatics 28, i333-i339 (2012).
67. Wala, J. A., et al. SvABA: genome-wide detection of structural variants and indels by local assembly. Genome Res 28, 581-591 (2018).
68. Layer, R. M., Chiang, C., Quinlan, A. R. & Hall, I. M. LUMPY: a probabilistic framework for structural variant discovery. Genome Biol 15, R84 (2014).

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for determining whether a prostate cancer patient should be treated with an androgen receptor (AR) independent therapy, comprising assaying a cell free DNA plasma sample from the patient to determine whether the sample comprises copy number gain or gain of function mutations to MYC, MYCN and/or AR, wherein the presence of said copy number gain or gain of function mutations to MYC, MYCN and/or AR indicates that the patient should be treated with an AR-independent therapy.

2. The method of claim 1, wherein the method is performed prior to the patient being treated with an AR inhibitor.

3. The method of claim 2, wherein the AR inhibitor is abiraterone, enzalutamide, apalutamide or darolutamide.

4. The method of claim 1, wherein the assaying determines a copy number gain of the AR gene.

5. The method of claim 1, wherein the assaying determines an AR upstream enhancer gain.

6. The method of claim 1, wherein the assaying determines a copy number gain of the AR gene body and an AR upstream enhancer gain.

7. The method of claim 1, wherein the assaying determines an AR mutation that encodes a H875Y substitution in the AR protein.

8. The method of claim 1, wherein the assaying determines an AR gene structural rearrangement (AR-GSR).

9. The method of claim 1, wherein the assaying determines the presence of copy number gain or gain of function mutations to MYC, MYCN and AR.

10. The method of claim 1, wherein the assaying further determines a mutation to TP53, PTEN, and/or RB1.

11. The method of claim 1, wherein the patient has an estimated ctDNA aneuploidy fraction of <=about 14.2%.

12. An assay for performing the method according to claim 1.

13. A system for performing the method according to claim 1.

14. A system for performing the assay of claim 12.
